(19) United States
(12) Patent Application Publication
(10) Pub. No.: US 2015/0203878 A1
(43) Pub. Date: Jul. 23, 2015

(54) PHA-PRODUCING GENETICALLY ENGINEERED MICROORGANISMS

(71) Applicant: HELMHOLTZ-ZENTRUM FUR INFEKTIONSFORSCHUNG GMBH, Brauschweig (DE)

(72) Inventors: Sagrario Arias Rivas, Leiden (NL); Mònica Bassas Galià, Sion (CH); Gabriella Molinari, Wolfenbuttel (DE); Kenneth Nigel Timmes, Chambesy (CH)

(73) Assignee: HELMHOLTZ-ZENTRUM FUR INFEKTIONSFORSCHUNG GMBH, Braunschweig (DE)

(21) Appl. No.: 14/391,939

(22) PCT Filed: Apr. 11, 2013
(86) PCT No.: PCT/EP2013/057630

§ 371 Date: Oct. 10, 2014

(30) Foreign Application Priority Data
Apr. 11, 2012 (EP) 12163787.0

(51) Int. Cl. C12P 7/62; C12N 9/10; C12N 15/78

(52) U.S. Cl. CPC C12P 7/625; C12N 15/78; C12N 9/1029

(57) ABSTRACT

The present invention is directed at genetically engineered form of a naturally PHA producing microorganism, which has an increased number of copies, compared to the wild type microorganism, of at least one gene coding a polyhydroxyalkanoate (PHA) synthase, wherein said increased number of copies provides a balanced overproduction of said PHA synthase, and eventually causing the microorganism to overproduce medium- or long-chain-length PHAs in an amount of at least 1.2 times compared to the wild type after 24 h, wherein the reference condition for assessing the overproduction is modified MM medium containing 15 mM sodium octanoate. The production of PHAs in the microorganism can in addition be favourably influenced by the inactivation of genes encoding for proteins involved in the degradation of PHA, resulting in an even increased production of the microorganism of this compound without a decline in the PHA content over time. The inventive microorganisms are useful in the commercial production of PHAs. The present invention further relates to a method for the production of PHA.

The present invention relates to the field of biosynthesis of polyhydroxyalkanoates (PHAs). In particular, the invention relates to a genetically engineered microorganism, which is stable on reproduction and has an increased number of copies, compared to the wild type microorganism, of at least one gene encoding a PHA synthase, wherein the genetic engineering causes the microorganism to overproduce medium- or long-chain-length PHAs.

PHAs belong to the type of polymers, which are biodegradable and bio-compatible plastic materials (polyesters of 3-hydroxy fatty acids) produced from renewable resources with a broad range for industrial and biomedical applications (Williams & Peoples, 1996, Chemtech 26: 38-44). PHAs are synthesized by a broad range of bacteria and have extensively been studied due to their potential use to substitute conventional petrochemical-based plastics to protect the environment from harmful effects of plastic wastes.

PHAs can be divided into two groups according to the lengths of their side chains and their biosynthetic pathways. Those with short side chains, such as PHB, a homopolymer of (R)-3-hydroxybutyric acid, are crystalline thermoplastics, whereas PHAs with longer side chains are more elastic. The former have been known for about 70 years (Lemoigne & Roukheiman, 1925, Ann Des Fermentation, 527-536), whereas the latter materials were discovered relatively recently (deSmet et al., 1983, 1, Bacterial. 154: 870-78). Before this designation, however, PHAs of microbial origin containing both (R)-3-hydroxybutyric acid units and longer side chain (R)-3-hydroxyacid units with 5 to 16 carbon atoms had been identified (Wallen & Roweder 1975, Environ, Sal. Technol. 8: 576-79). A number of bacteria which produce copolymers of (R)-3-hydroxybutyric acid and one or more long side chain hydroxy acid units containing from 5 to 16 carbon atoms have been identified (Steinbuchel & Wiese, 1992, Appl. Microbial. Biotechnol. 37: 691-97; Valentin et al., 1992, Appl. Microbiol. Biotechnol, 36: 507-14; Valentin et al., Appl. Microbiol, Biotechnol. 1994, 40: 710-16; Abe et al., 1994, Int. 3. Biol, Macromol, 16: 115-19; Lee et al., 1995, Appl. Microbiol, Biotechnol. 42: 901-09; Kato et al., 1996, Appl. Microbial!. Biotechnol. 45: 363-70; Valentin et al., 1996, Appl. Microbiol, Biotechnol, 46: 261-67; and U.S. Pat. No. 4,876,331). These co-polymers can be referred to as PHB-co-HX (wherein X is a 3-hydroxy alkanoate or alkenoate of 6 or more carbon atoms). A useful example of a specific two-component copolymer is PHB-co-3-hydroxyhexanoate (PHB-co-3HH) (Brandl et al., 1989, Int, 3, Biol, Macromol, 11: 49-45; Amos & McInerey, 1991, Arch. Microbiol. 155: 103-06; U.S. Pat. No. 5,292,860).

Although PHAs have been extensively studied because of their potential use as a renewable resource for biodegradable thermoplastics and biopolymers (as mentioned above) and have been commercially developed and marketed (Hrabak, 1992, FEMS Microbial. Rev, 103: 251-256), their production costs are much higher than those of conventional petrochemical-based plastics. This represents a major obstacle to their wider use (Choi & Lee, 1997, Bioprocess Eng. 17: 335-342). As described above, many bacteria produce PHAs, e.g. Alcaligenes eutrophus, Alcaligenes latus, Azotobarter vinlandii, Pseudomonas acitophila, Pseudomonas oleovarans, Escherichia coil, Rhodococcus eutropha, Chromobacterium violaceum, Chromatium vinosum, Alcanivorax borcumensis, etc. All these PHA producing bacteria are known in the art to produce intracellular PHA and accumulate it in PHA granules (Steinbüchel, 1991, Biomaterials, pp. 123-213).

The main aspects, which render PHA production expensive and therefore unfavorable as compared to petrochemical-based plastics, are that it is difficult to produce the material in high yield and to recover the produced PHA from within the bacterial cells where it is accumulated. In order to reduce the total production costs of PI-IA, the development of an efficient recovery process was considered to be necessary generally aiming at cell disruption (Lee, 1996, Biotech, Bio-eng. 49: 144) by i) an appropriate solvent, ii) hypochlorite extraction of PHA and/or iii) digestion of non-PHA cellular materials.

At an industrial scale, the available microorganisms still provide relatively little PHA, which renders the production of PHA with these microorganisms economically non-feasible. For example, when the wild type cells of Pseudomonas putida U is cultivated in modified MM media containing sodium octanoate (15 mM) as a carbon source, only 24.4% of PHA accumulated in the microorganism during the first 24 hours. All methods for microorganism based PHA production known in the art require large amounts of water during the production and in addition chemical reagents and/or enzymes for their recovery, which is an obstacle to reducing the production costs. Therefore, alternative strategies for PHA production are in urgent need.

In addition to overall low PHA production by microorganism, the amount of accumulated PHA at a certain stage of the cultivation starts to decline. The reason for this decline can be traced back to the fact, that the microorganisms produce PHA as a food storage material, which serves the bacteria as a swift source of energy and reducing power in changing environments. All free-living microorganisms practice some kind of carbon resource management to the extent that is possible. Whereas many animals and plants generally regulate carbon uptake to match metabolic needs, other organisms, particularly opportunistic environmental microbes subjected to widely fluctuating carbon availability can capture excess carbon and manage its utilization as through consumption and growth on one hand, and conservation by conversion to storage polymers on the other. Interconversions between readily metabolizable and more inert intracellular, and to some extent also extracellular storage products, are central to this mechanism. Even organisms that regulate carbon uptake exploit such interconversions for fine-tuning of their carbon management to optimize their cellular metabolic networks and organismal ecophysiological processes.

As mentioned above, PHAs are widely exploited storage products in the microbial world. To allow for the utilisation of the carbon stored as PHA in the microorganism, it is vital for the organism, that the PHA can be reconverted to hydroxyalkanoates (i.e. the monomers) when the microorganism is in need of extra carbon sources. Responsible for this reconversion of the polymer to individual monomer units are PHA depolymerases.

Since the microorganism contains both types of proteins responsible for PHA production and degradation, one key issue for the organism to ensure its survival and prosperity is the regulation of the relative amounts of PHA synthase and PHA depolymerase, which are determined by their regulated production (Uchino et al., 2007; Ren et al., 2009a; and de Eugenio et al., 2010a, 2010b). Thus far, however, the factors controlling the processes of polymerization and depolymerization are poorly understood. For example, the mere knock-out of PHA depolymerases in Pseudomonas strains did not result in improved accumulation of PHA (Huisman et al., 1991; Solaiman et al., 2003). Thus, it turns out that the mere silencing of genes responsible for PHA depolymerization is not sufficient to effectively increase the PHA content in microorganisms.

A different approach to increase the PHA production in a microorganism has been to manipulate the PHA synthases responsible in the microorganism for the production of PHAs. For example, the metabolic engineering of PHA genes was found as a good strategy for the scale up of medium-chain-length PHA production. Previous studies attempted to increase PHA yields in Pseudomonas putida by an overexpression of phaC1 (kraak et al., 1997; Prieto et al., 1999; Conte et al., 2006; Kim et al, 2006; Ren et al., 2009b). However, these studies encountered the problem that phaC-containing plasmids are lost when they are not vital for growth and impose detrimental effects in the cells. As a result, the modified microorganisms were not stable upon reproduction and lost the genetic information responsible for the overproduction of PHA. In other cases, less PHA accumulation was attained, since high induction of a promoter did not always entail high activity of the gene product (Diederich et al., 1994; Ren et el., 2009).

The reason for these attempts being unsuccessful may be found in the many different proteins involved in the production, storage and degradation of PHA in the microorganism. Most microorganisms have more than one PHA synthase, so increasing the number of genetic copies of one synthase may deplete the microorganism from metabolites important for the production of other PHA synthases resulting in only a modest improvement of PHA synthesis in the microorganism.

In addition, phasines play an important role in PHA-granule stabilisation in the microorganism. For example, phasines control the number and size of the Pt-IA granules (Grage et al., 1999) creating an interphase between the cytoplasm and the hydrophobic core of the PHA granule, thus, preventing the individual granules from coalescing (Steinbüchel et al., 1995; York et al., 2002). It also has been suggested that the phasin PhaF and some global transcriptional factors as Crc) are important for the regulation of the PhaC activity (Prieto et al., 1999b; Castaneda et al., 2000; Kessler & Witholt, 2001 ; Hoffmann & Rehm, 2005; Ren et al., 2010). Recent studies in P. putida KT2440 (Galan et al., 2011) have demonstrated that PhaF plays an important role in the granule segregation, and even more, that the lack of this phasin entails the agglomeration of these inclusion bodies in the cytoplasm.

It therefore represents a considerable challenge to modify microorganisms such that they overproduce PHA to a significant extent, while at the same time ensuring that the modification leading to overproduction is stable upon reproduction of the microorganisms and that no proteins involved in the handling of the microorganism of PHA are affected so severely that the desired result is overcompensated. With most approaches pursued so far it has in addition been difficult to find the precise point in time where PHA accumulation is at its peak, and to recover the PHA before PHA decomposition sets in.

One approach, which has been successful to some extent in this regard has been described in WO 2007/017270 A1, wherein Alcanivorax borcumensis has been modified by silencing the tesB-like gene. This gene encodes for a thioesterase, which converts the (R)-3-OH-Acyl-CoA intermediate to the corresponding acid. This is an important side reaction, depleting the microorganism from an intermediate vital for PHA synthesis. While this approach has been proven successful to some extent in that a higher accumulation of PHA was achieved, it remains to be seen whether the modified microorganism has the required stability to allow for successful implementation into an industrial scale production of PHA.

Another approach has been to overexpress PHA synthases like phaC1 and phaC2 in P. putida KCTC1639, which has been described by Kim et al (2006, Biotechnol. Prog. 22: 1541-1546). In this investigation, additional copies of phaC1 and phaC2 genes were introduced into the microorganism via plasmids, wherein the genes were not under the control of a promoter. Kim et al, describe that the PHA synthese activity in the modified microorganism was more than 1.6 fold the activity of the wild type. While in case of the microorganism overexpressing phaC1 an increased PHA production (up to about 0.8 gl⁻¹) could be observed, the microorganism overexpressing phaC2 did not show an increase of PHA production over the wild type. This observation is likely due to the formation of non-active forms of phaC2 synthase.

A yet further approach was to insert PHA synthase genes into microorganisms, which in their wild type form do not produce PHA. For example WO 99/14313, DE 44 17 169 A1 or Qi et al. (1997, FEMS Microbiol. Lett, 157: 155-162) describe the introduction of PHA synthase genes into E coli. However, in these engineered microorganisms, the yield of PHA produced was very low, making them unsuitable for the industrial production of PHAs.

Finally, Cai et al. (2009, Biores. Technol. 100: 2265-2270) has reported the enhanced production of PHA via knock-out of the PHA depolymerase gene in P. putida KT 2442. In this study, an increase of PHA production could be observed, when the microorganism was cultivated in the presence of high carbon source concentrations such as 12 gl⁻¹.

Despite of these advancements, there remains a need for genetically modified microorganisms, which have an increased overproduction of PHA and at the same time are stable upon reproduction in that they do not loose the genetic information inserted for this purpose. The present application addresses this need.

### BRIEF DESCRIPTION OF THE INVENTION

One aim of the present application is to provide a genetically engineered microorganism wherein the genetic information responsible for the overproduction of medium- or long-chain-length PHAs in the microorganism is stable upon reproduction. Another aim of the present invention is to modify the microorganism such, that the decline of PHA after a certain exposure time to cultivation medium is avoided and at the same time the percentage of PHA accumulation is increased. Yet, another aim of the present application is to modify the microorganism such, that significant PHA degradation, once the PHA has been accumulated, is prevented.

The present invention is based on the finding that these goals can be achieved by modifying PHA-producing microorganisms such that they have an increased number of copies compared to the wild type microorganism, of at least one gene encoding a PHA synthase. Preferably the gene present in additional copies encodes for phaC2 or homologues thereof. The wild type microorganism, as this term is used in the present application, means the typical form of the microorganism as it occurs in nature. Preferably, the wild type microorganism, in its native form, comprises at least one gene encoding a PHA synthase.

The term “homolog” is defined in the practice of the present application as a protein or peptide of substantially the same function but a different, though similar structure and sequence of a parent peptide. In the context of the present application the terms “percent homology” and “sequence similarity” are used interchangeably. In the practice of the present application is preferred that the homolog should have at least 40%, 50%, 60%, 70%, 75%, 80%, 85%, 90% and most preferably at least 95% sequence identity to the parent peptide. A preferred non-limiting example of a mathematical algorithm used for the comparison of two sequences is the algorithm of Karlin et al. (1993, PNAS 90: 5873-5877). Such algorithm is incorporated into the NBLAST program, which can be used to identify sequences having the desired identity to nucleic acid sequences of the invention.

Thus, one primary aspect of the present application is a genetically engineered form of a naturally PHA producing microorganism, which has an increased number of copies compared to the wild type microorganism of at least one gene encoding a PHA synthase, wherein said increased number of copies provides a balanced overproduction of said PHA synthase and eventually causes the microorganism to overproduce medium- or long-chain-length PHAs in an amount of at least 1.2 times compared to the wild type after 24 h, wherein the reference condition for assessing the overproduction is modified MM medium containing 15 mM sodium octanoate. In a preferred embodiment, the genetically engineered microorganism is stable upon reproduction and preferably has one additional copy compared to the wild type microorganism of the at least one gene encoding a PHA synthase,

It has unexpectedly been discovered, that these genetically modified microorganisms allow for the highly cost efficient production of PHA from cheap and readily available feedstocks including fatty acid derived from vegetable fats and oils. The inventive microorganisms have been observed to provide high PHA peak concentration, which is reached, depending on the cultivation conditions, in some cases even after only 24 h. Moreover the inventive microorganisms exhibit a high genetic stability and fusion of individual PHA granules in the microorganism to form a single PHA granule. This in turn greatly simplifies the recovery of the PHA from the microorganisms, because they can be extracted with non-chlorinated solvents such as acetone with yields comparable to the extraction with chlorinated solvents.

The term “genetically engineered” (or genetically modified) means an artificial manipulation of a microorganism of the invention, its gene(s) and/or gene product(s) (polypeptide).

Preferably, the inventive microorganism is stable upon reproduction. “Stable upon reproduction” as this term has to be understood in the practice of the present application) means, that the organism maintains the genetic information upon multiple (such as e.g. 5 or more) reproduction cycles and that the genetic information is not lost.

As stated above, the inventive microorganisms are preferably stable upon reproduction which means that the genetic modification is maintained in the microorganism on reproduction and/or cultivation. In addition to such stability it is preferred that the microorganism does not require the pressure of an antibiotic to preserve the genetic modification. Such microorganisms are highly advantageous for PHA production, since addition of antibiotic can be omitted and thus the risk to contaminate PHA with antibiotics is eliminated. In a preferred embodiment of the present application the inventive microorganism thus maintains its genetic modification during reproduction and/or cultivation independent on the presence or absence of an antibiotic.

The term “balanced overexpression” means that the overexpression is such that the protein produced by overexpression is produced in less than the amount expectable from the increased number of copies. For example, if the wild type comprises one copy of the gene and the genetically modified microorganism comprises two copies, one can expect the genetically modified microorganism to produce about twice as much of the protein compared to the wild type. The amount of protein can be estimated from the intrinsic PHA synthase activity in the growth phase of the microorganism. The term balanced overexpression means that the overexpression preferably only leads to an increase of the intrinsic PHA synthase activity in the growth phase after 24 h of up to 0.6 times, preferably up to 0.5 times, more preferably up to 035 times and most preferably up to 0.2 times relative to wild type microorganism.

By using a “balanced overexpression” it is ensured that no substantial amounts of inactive proteins are formed. For example, extensive (or unbalanced) overexpression of proteins may lead to the formation of inclusion bodies which comprise the protein in a non-active form and as undissolved protein. Hence, despite of an overexpression of the protein, no improved protein activity can be observed. One method to ensure a balanced overexpression is the use of a leaky promoter system, which allows a suppressed protein production even in the absence of an inducer.

In a preferred embodiment of the present application, the overproduction is at least partially caused by the increased number of copies of the at least one gene encoding a PHA synthase, In a further preferred embodiment, the gene of which the microorganism contains more than one copy is the gene encoding for the PhaC2 synthase. In the practice of the present application it has been found, that the insertion of multiple copies of the phaC2 gene or homologs thereof is associated with beneficial effects, in particular that the hyperexpression of a phaC2 involves changes in the morphology of the PHA granules, which appear to coalesce together, especially during the exponential growth phase.

Moreover, it is believed that the insertion of multiple copies of PhaC2 synthase gene under the control of a leaky promoter positively affects other proteins in volved in PHA metabolism so that the overall PHA production and storage system of the microorganism is not negatively affected.

In a further preferred embodiment, the expression of PHA synthase gene is thus regulated by a leaky promoter system. A leaky promoter system allows for the transcription of the promoter controlled gene, albeit with suppressed efficiency compared to the system in which the promoter is activated with a corresponding activator. The leaky promoter system is preferably a protein-based promoter system and more preferably a T7 polymerase/T7 polymerase promoter system. In an even more preferred embodiment, the production of the 17 polymerase in this T7 polymerase/T7 polymerase promoter system comprises an inducer capable to induce the formation of T7 polymerase upon exposure to a small molecule. Such system has the added benefit that it is possible to selectively trigger the production of T7 polymerase by the addition of a small molecule resulting in an induction of the formation of the T7 polymerase. This in turn then triggers the PHA synthase production. In a particular preferred embodiment, the small molecule is 3-methyl-benzoate.

One highly preferred inventive genetically engineered form of an naturally PHA producing microorganism is of the genus Pseudomonas as deposited under DSM 26224 with the Leibnitz Institute DSMZ German collection of microorganisms and cell cultures which will in the following be designated as Pot) 10-33.

It is further preferred in the practice of the present application that genetically engineered microorganisms, which in addition to an increased number of copies, compared to the wild type microorganism, of at least one gene encoding a PHA synthase contains at least one modification in at least one gene encoding a protein involved in the degradation of PHA. Such a combination of modifications in a microorganism has been found to result in a synergistic effect with regard to the observed PHA accumulation. In a preferred embodiment, the at least one modification in at least one gene encoding a protein involved in the degradation of PHA in said microorganism causes complete or partial inactivation of said gene, preferably complete inactivation of the gene. Such microorganisms are also called knock-out microorganisms for the respective gene.

The knock-out mutants can be prepared by any suitable process known to the skilled practitioner. It is preferred however, that complete or partial inactivation of the gene is achieved by a double recombinant crossover-event approach.

In a particularly preferred embodiment, the protein involved in the degradation of PHA is a PHA depolymerase, preferably PhaZ or a homologue thereof. In addition, it is preferred, that the genetically engineered microorganism, wherein the gene encoding a protein involved in the degradation of PHA contains at least one modification, only contains a single gene encoding a protein involved in the degradation of PHA in said microorganisms, i.e, only the gene which is modified. In other words, it is preferred that the microorganism does not contain any other enzymes which can replace the enzyme involved in the degradation of PHA in said microorganism.

One highly preferred inventive genetically engineered form of an naturally PHA producing microorganism comprising both, multiple copies of a gene encoding a PHA synthase and a deactivated phaZ gene, is of the genus Pseudomonas as deposited under DSM 26225 with the Leibnitz Institute DSMZ German collection of microorganisms and cell cultures. This microorganism will in the following be designated as PpU 10-33-ΔphaZ

A typically polyester of hydroxy acid units (PHA) contains side chain hydroxy acid units [(R)-3-hydroxy acid units] from 5 to 16 carbon atoms. The term “long-chain-length PHA” is intended to encompass PHAs containing at least 12, preferably at least 14 carbon atoms per monomer (molecule), whereas 5 to 12 carbon atoms are intended to be meant by “medium-chain-length PHAs” in the practice of the invention. In a preferred embodiment, the genetically engineered microorganism overproduces medium-chain-length PHAs.

In a particularly preferred embodiment of the present application, the genetically engineered microorganism is caused by the genetic engineering, i.e. for example the insertion of an increased number of copies compared to the wild type of at least one gene encoding a PHA synthase and/or the insertion of at least one modification in at least one gene encoding a protein involved in the degradation of PHA in said microorganism, to overproduce PHA in an amount of at least 1.2 times, preferably at least 1.5 times and in particular at least 2 times by weight) compared to the wild type after 24 h, wherein the reference condition for assessing the overproduction is modified MM medium containing 15 mM sodium octanoate.

The microorganism, which forms the basis of the genetically engineered microorganism of the present application, is not restricted by any means, except that the microorganism must possess at least one gene encoding for a PHA synthase. Preferably, the microorganism should also have at least one gene, more preferably a single gene, encoding for a protein involved in the degradation of PHA in said microorganism.

The inventive microorganism in accordance with the present application is preferably selected from the group of PHA producing bacteria, in particular from Pseudomonas putida, Pseudomonas aeruginosa, Pseudomonas syringae, Pseudomonas fluorescens, Pseudomonas acitophila, Pseudomonas olevarans, Idiomarina Alcanivorax borkumensis Acinetobacter sp., Caulobacter crescentus, Alcaligenes eutrophus, Alcaligenes latus, Azotobacter vinlandii, Rhodococcus eutropha, Chromobacterium violaceum or Chromatium vinosum. An especially preferred microorganism according to the present invention is a Pseudomonas putida strain, more preferably Pseudomonas putida U.

It has been observed, that the microorganisms of the present application exhibit an overproduction of PHA synthases in the absence of an inducer molecule. Un expectedly, the production of PHA by the non-induced microorganisms matched or even exceeded the PHA production of identical microorganisms which were treated with an inducer. This suggests that the induced microorganisms can over-shoot the optimum amount of overexpressed PHA synthase, which results in the formation of non-active forms of the synthase such as inclusion bodies or non-dissolved forms. Therefore, a further aspect of the present application is directed at genetically engineered microorganisms as described above, wherein the microorganisms are capable to produce PHA without the addition of an inducer molecule. This has advantages for the industrial scale production of PHA as it is possible to omit expensive inducer and potential contamination risks from the production process.

It has further been unexpectedly observed, that the microorganisms of the pre sent application produce PHA with a different morphology compared to the wild type, in that the individual cells produce a reduced number or even only a single granule of PHA. Therefore a further aspect of the present application is directed at genetically engineered microorganism as described above, wherein the microorganism is capable to produce a reduced number of intercellular PHA granules per microorganism compared to wild type cells, preferably in the form of a single intercellular PHA granule. The formation of a single granule is believed to be associated with a reduced amount of PHA stabilizing enzymes, which simplifies PHA isolation and purification.

It has also been unexpectedly observed, that the microorganisms of the present application produce PHA faster and in some cases maintain a high level of accumulated PHA over a long period. Therefore a further aspect of the present application is directed at genetically engineered microorganism as described above, wherein the microorganism is capable to produce a maximum content of PHA after 24 h upon exposure to modified MM medium containing sodium octanoate and preferably is also capable to maintain a PHA content, which is in a range of 20% by weight of the maximum PHA content, for a time of at least 48 h after the initial 24 h accumulation period, wherein the reference condition for assessing the PHA production is modified MM medium containing 15 mM sodium octanoate.

A further aspect of the present invention relates to a method for producing PHAs comprising the following steps:

a. Cultivating a microorganism or a cell of the invention and

b. recovering PHA from the culture medium.

Standard methods for cultivating a microorganism or a cell under suitable conditions are well-known in the art. See for example below under examples, materials and also Sambrook & Russell (2001). PHA can be isolated from the culture medium by conventional procedures including separating the cells from the medium by centrifugation or filtration, precipitating or filtrating the components (PHA), followed by purification, e.g. by chromatographic procedures, e.g. ion exchange, chromatography, affinity chromatography or similar art recognized procedures.

It is preferred that the PHA in the above mentioned process is recovered by extraction with a ketone having 3 to 8 carbon atoms, preferably with acetone. Independent of the extraction solvent, the extraction is preferably carried out at a temperature of 60° C. or less, preferably at 20 to 40° C..

In a particularly preferred embodiment of the present application, the method does not involve or require the addition of an inducer molecule to initiate PHA overproduction and/or overproduction of PHA synthases. In addition, in the practice of the present application it is not necessary to cultivate the inventive microorganisms in the presence of an antibiotic, as it has unexpectedly been found that the microorganisms are stable with regard to the introduced modifications even in the absence of an antibiotic. Such antibiotics include without limitation Tellurite, Rifampicin and Kanamycin.

As the carbon feedstock for the above described process It is possible to use readily available and cheap fatty acids derivable from vegetable fats and oils. Preferred examples of such fatty acids include saturated carboxylic acids such as hexanoic, heptanoic, octanoic and decenoic acid, and unsaturated fatty acids such as 1-unclecenoic acid, oleic acid or linoleic acid. In addition it is possible to use polyhydric alcohols as the feedstock such as preferably glycerol.

Another aspect of the invention relates to the use of a microorganism, a nucleic acid, a vector and/or a cell of the invention for the overproduction of PHAs, especially medium- and/or long-chain-length PHAs.

### BRIEF DESCRIPTION OF THE FIGURES

FIG. 1 Electron micrographs of PpU (a-c); PpU 10-33 non-induced (d-f) and PpU 10-33 induced cells (g-i); ΔphaZ-PpU10-33 non-induced (j-l) and induced (m-o) cells. Cultures were grown in modified MM containing 35 mM sodium octanoate as a carbon source (given in two pulses of 15 mM and 20 mM) and sampled at 31 h (a, d, g, j, m), 48 h (b, e, h, k, n) and 72 h (c, f, i, l, o).

FIG. 2. Expression of pha genes and PHA accumulation in P. Putida U. Each panel shows normalized fold-increased in expression of the pha genes in PpU (first bar for each number), PpU 10-33 non-induced (second bar for each number in (a) and (c)) and Poll 10-33 induced (third bar for each number in (a) and (c)), ΔphaZ-PpU10-33 non-induced (second bar for each number in (b)) and ΔphaZ-PpU10-33 induced (second bar for each number in (b)). The PHA content (g l⁻¹) is also shown in a straight line with dots (PpU), lower broken line with triangles (PpU 10-33 induced), dots (PpU 10-33 non-induced), upper broken line with triangles (ΔphaZ-PpU10-33 non-induced) and broken line with rectangles (ΔphaZ-PpU10-33 induced) in graph (c).

FIG. 3. Genetic organization of the bipartite system for hyper-expression of phaC2 in P. putida U. The diagram shows the two vectors, pCNB1mini-Tn5 xylS/Pm::T7pol and pUTminiTn5-Tel-T7phaC2, integrated into the chromosome.

FIG. 4. PHA production overtime in the wild type PpU (squares), as well as the genetically engineered constructs PpU 10-33 non-induced (filled circles), PpU induced (open circles), ΔphaZ-PpU10-33 non-induced (filled triangles) and ΔphaZ-PpU10-33 induced (open triangles).

FIG. 5. Biomass and PHA yields of PpU and PpU 10-33-ΔphaZ when were cultivated in MM+0.1% YE medium and octanoate (20 mM) as substrate, with and without the corresponding antibiotics. Results are means of duplicates.

in the following, the present application is further illustrated by way of examples, which however are not intended to limit the scope of the present application by any means.

### EXAMPLES

Experimental Procedures

Microorganisms and vectors, Bacterial strains, mutants and plasmids used in this work are summarized in Annex 1.

Culture Media Conditions

Unless otherwise stated, E. coil and P. putida strains were cultured in Luria Miller Broth (LB) and incubated at 37° C. and 30° C., respectively. Where required, antibiotics were added to media as follows: rifampicin (Rf, 20 μg in solid, or 5 μg ml⁻¹ in liquid media), kanamycin (Km, 25 μg ml⁻¹ in solid, or 12.5 μg ml⁻¹ in liquid media), ampicillin (Ap, 100 μg ml⁻¹), tellurite (Tel, 100 μg gentamicin (Gm, 30 μg ml⁻¹) chloramphenicol (Cm, 30 μg ml⁻¹), Isopropyl-β-D-thiogalactopyranosid (IPTG, 70 μM) and 5-brorno-4-chloro-3-indolyl-beta-D-galactopyranoside (XGal, 34 μg ml⁻¹).

DNA Manipulations

All genetic procedures were performed as described by Sambrook & Russell (2001). Genomic and plasmid DNA extraction, agarose gel purification and PCR cleaning were carried out using the corresponding Qiagen kits (Germany), as per the manufacturers' instructions. All DNA modifying enzymes (restriction endonucleases, DNA ligase, alkaline phosphatase, etc.) used in this work were purchased from NEB (Massachusetts, USA). Polymerase chain reactions (PCR) were performed in an Eppendorf vapo.protect Thermal Cycler (Germany). The 50 μl PCR reaction mixtures consisted of 2 μl of the diluted genomic DNA (50 μg ml⁻¹), 1 x PCR buffer and 2 mM MgCl₂ (PROMEGA Co., USA), 0.2 μM of each primer (Eurofins mgw Operon) 0.2 mM dNIPs (Amersham, GE HealthCare, UK), 1.25 U Go-Taq Hot Start Polymerase (PROMEGA Co., USA). PCR cycling conditions were: an initial step at 96° C. 10 min followed by 30 cycles of 96° C. 30 s-−60° C. 30 s 72° C. 1 min, with a final extension at 72° C. 5 min. Plasmid transfer to Pseudomonas strains was made by triparental conjugation experiments (Selvaraj & Iyer, 1983; Herrero et al., 1990). Briefly, the E. coli 18λpir donor strain harbouring the suicide plasmid pCNB1mini-Tn5 xylSPm::T7pol or pUTminiTn5-Tel-phaC2, the E. coli RK600 helper strain, and the Pseudomonas recipient strain, were cultivated separately for 8 h, mixed in the ratio 0.75:1:2, and washed twice with LB. The suspension was collected on a nitrocellulose filter and incubated overnight on an LB plate at 30° C. Bacteria growing on the filters were then re-suspended in 3 ml of sterile saline solution (NaCl 0.9%) and serial dilutions plated on LB agar supplemented with the corresponding selection antibiotics. Plates were incubated overnight at 30° C. and transconjugants clones developing on the plates were confirmed by PCR.

DNA Sequencing

PCR reactions for sequencing were performed using either a set of specific oligonucleotides or the universal primers M13F and M13R (Annex 3). The 10 μl reaction mixtures consisted of 6-12 ng of the purified PCR product (or 200-300 ng plasmid), 2 μl BigDye Ready Reaction Mix, 1 μl of BigDye sequencing buffer and 1 μl of the specific primer (25 μM). The cycling conditions included: an initial step at 96° C. I 1 min, followed by 25 cycles of 96° C. 20 s 52° C.-58° C. 20 s 60° C. 4 min, with a final extension step at 60° C. 1 min. Nucleotide sequences were determined using the dideoxy-chain termination method (Big Dye Terminator v3 .1 Kit, Applied Biosystems, Foster City, USA). PCR products were purified using the Qiagen DyeEx 2.0 Spin Kit (Germany). Pellets were resuspended in 20 μl water and loaded onto the ABI PRISM 3130 Genetic Analyser (Applied Biosystems, California, USA). Partial sequences obtained were aligned with known sequences in the non-redundant nucleotide databases (www.ncbi.nlm.nih.gov). Identification of potential tanscriptional promoter regions and terminators was made using the Softberry, (http://linux1.softberry.com/cgi-bin/programs/gfindb/bprom.pl), Prom-Scan (http://molbiol-tools.ca/promscan/), and POBG online (http://www.fruitfly.org/seq_tools/promoter.html); and Arnold (http://rna.igmors.u-psud.fr/toolbox/amold/index.php#Results) bioinformatics tools.

Design and Construction of the phaC2 Hyper-Expression Strain PpU 10-33

PpU 10-33 is a Pseudomonas putida U derivative in which the extra copy of the phaC2 gene expression is driven by the T7 polymerase promoter: T7 polymerase system. It consists of two chromosomally-integrated cassettes: one containing the phaC2 gene expressed from the T7 polymerase promoter, and another containing the T7 polymerase gene expressed from the Pm promoter and regulated by the cognate benzoate/toluate-inducible XylS regulator derived from the TOL plasmid. The phaC2 cassette was constructed as follows: The phaC2 gene of P. putida U was excised from the pBBR1MCS-3-phaC2 plasmid (Arias et al. 2008), cloned into the pUC18NotI/T7 vector (Herrero et al., 1993), and the correct orientation of the gene confirmed by sequencing. The phaC2 gene and the T7 promoter were then transferred as a cassette into the pUTminiTn5-Tel vector (Sanchez-Romero et al. 1998). First, the miniTn5 derivative pCNB 1 xy/S/Pm::T7pol, was transferred to P. putida U by filter-mating and selected by the Km selection marker (Harayarna et at, 1989; Herrero et al. 1993). Since integration of the transposon in the genome is essentially random, and different sites of insertion can markedly influence transcription levels of inserted genes, a pool of approximately 100 transconjugants was prepared for the second transfer. A 5ml LB culture of this pool was incubated for 3 h (30° C., 180 rpm), and used a pool of recipients for transfer of the pUTmini-Tn5-Tel-T7phaC2 construct. Transconjugants were readily scored by the black colour they display when they transform the tellurite (selection (selection marker), and subsequently confirmed by PCR. The final recipients varying in insertion sites of both cassettes were subsequently scored for levels of PhaC2 and PHA (Results) and the best selected and designated PpU 10-33.

Knock-out of phaZ in PpU 10-33 and Complementation

Deletion of the phaZ gene was accomplished by using a method described by Quant & Hynes, 1983; Donnenberg & Kaper, 1991, involving a double-recombination event and selection of the required mutant by expression of the lethal sacB gene. First, a DNA containing the ORFs adjacent to the phaZ gene, encoding the PheC1 and PhaC2 synthases, was synthesized by GENEART AG (Germany), was and subsequently cloned into the plQ200SK vector containing the Gm and Sac8 selection markers. The hybrid plasmid was then introduced by triparental mating into the PpU 10-33 strain. Transconjugants in which the plas mid was integrated into the chromosome by a single crossover, were selected on Gm-plus km and Tel-containing plates and confirmed by PCR. Deletion mutants resulting from the second recombination were subsequently selected on LB plates with 10% sucrose, scored for sensitivity to Gm, and further analyzed by PCR to confirm the position and extent of the deletion. For this, two different primer sets, annealing either outside or inside of the fragment used for the homologous recombination were used, namely PhaC1-check-F PhaC2-check-R and RT-phaZ F_PpU/RT-phaZ R_PpU, respectively. One deletion mutant was selected and designated ΔphaZ PpU 10-33, For complementation of the deletion mutant, the phaZ gene (921 bp) was amplified by PCR (phaZ-F-KpnI lphaZ-R-XbaI) and cloned into the pBBR1MCS-5 vector. Transcojugants were selected for their Gm resistance and further confirmed by PCR.

Fluorescence Microscopy

One ml of culture was mixed with 2 drops of a Nile red solution in dimethyisulfoxide (0.25 mg ml⁻¹) in a 1.5 ml Eppendorf tube and centrifuged at 6,500 rpm at 4° C., 5 min. Pellets were washed twice with 2 ml MgCl₂ (10 mM), resuspended in 500 μl of the solution and 5-10 μl of the cell suspension mounted on a microscopic slide. The presence and morphology of PHA granules was visualized with a ZEISS Axio Imager A1 epiflourescence microscope equipped with a Cy3 filter (EX BP 550/25, BS FT 570, EM BP 605/70) (ZEISS, Jena, Germany) and the AxioVision rel 4.6.3 software (Zeiss Imaging solutions GmbH, Germany). Cells were imaged at an exposure time of 1.1 s (Bassas et ac 2009).

Transmission Electron Microscopy

Bacteria were fixed with 2% glutaraldehyde and 5% formaldehyde in the growth medium at 4° C., washed with cacodylate buffer (0.1 M cacodylate, 0.01 M CaC1₂, 0.01 M MgCl₂, 0.09 M sucrose, pH 6.9), and osmificated with 1% aqueous osmium for 1 h at room temperature. Samples were then dehydrated in a graded series of acetone (10%, 30%, 50%, 70%, 90%, and 100%) for 30 min at each step. The 70% acetone dehydratation step included 2% uranyl acetate and was carried out overnight. Samples were infiltrated with an epoxy resin according to the Spurr formula for hard resin, a low-viscosity epoxy resin embedding medium for electron microscope (Spurr, 1969). Infiltration with pure resin was done for several days. Ultrathin sections were cut with a diamond knife, counterstained with uranyl acetate and lead citrate, and examined in a TEM910 transmission electron microscope (Carl Zeiss, Germany) at an acceleration voltage of 80 kV. Images were taken at calibrated magnifications using a line replica and recorded digitally with a Slow-Scan CCD-Camera (ProScari, 1024x1024, Scheuring, Germany) with ITEM-Software (Olympus Soft Imaging Solutions, Germany).

RNA manipulations

Samples (3 ml) were taken from cultures through the growth phase (4 h, 7 h, 24 h, 27 h, 31 h, 48 h and 55 h) and immediately mixed with an equal volume of RNA protect Buffer (Qiagen, Germany). After incubation for 5 min at room temperature, suspensions were centrifuged at 13,000 rpm, the supernatant fluids discarded and pellets kept at −80° C. Total RNA was extracted using the RNeasy mini kit (Qiagen, Germany) including the DNase treatment, as per the manufacturer's protocol. Finally, RNA was eluted in 100 μL of free-RNase water and kept at −80° C. The integrity of the RNA was assessed by electrophoresis in formaldehyde agarose gels and the concentration and purity determined spectrophotometrically (Spectrophotometer ND-100, peQlab-biotechnologie GmbH, Germany).

cDNA was carried in 20 μl reactions using 10 μg of total RNA and Random Primers. All reagents (included Superscript III RT), were purchased from Invitrogen (USA) and reactions performed according manufacturer's protocols. Samples in which Superscript III RT was not added were used as negative controls. After cDNA synthesis, the remaining RNA was precipitated with 1 M NaOH, incubated at 65° C. 10 min, followed by 10 min at 25° C. Immediately, the reaction was equilibrated with KCl 1 M. The resultant cDNA was then purified using the PCR purification kit (Qiagen) and the concentration and purity was measured with the Spectrophotometer cDNAs were diluted with DEPC water to 100 ng μl⁻¹ and kept at 4° C.

Relative RT-PCR Assay

Oligonucleotides used for the RT-PCR assays (Eurofins mgw Operon, Germany) were designed with the help of the Primer3 (http://frodo.wi.mit.edu/primer3/) and Oligo Calc (http://www.basic.northwestern.edu/biotools/oligocalc.html) bio-informatic tool and are summarized in Annex 2. Each set was designed to have similar G+C contents, and thus similar annealing temperatures (about 60° C.), an amplicon product size no longer than 300 bp, and absence of predicted hairpin loops, duplexes or primer-dimmer formations, The MIQE guidelines for the experimental design were followed (Bustin et al., 2009). First, each set of primers was assayed for optimal PCR conditions, and annealing temperature and primer concentrations were established using a standard set of samples (genomic DNA) as templates. Primer specificity was determined by melt curve analysis and gel visualization of the amplicon bands. Primers efficiency was determined with a pool of cDNAs and underwent to serial 4-folds dilutions series over five points to perform the standard curve. A standard PCR protocol was performed in triplicate for each dilution. In all cases, efficiencies were measured in the range between 89% and 100%. For this assay the CFX96™ real-time PCR detection system (Bio-Rad, USA) and the CFX Manager software (version 1,5.534.0511, Bio-Rad) was used. The choice of appropriate reference genes for data normalization was carried out using the geNorm method existing in the CFS software and taking into consideration the target stability between the different experimental conditions and the time points, considering good values a coefficient variance and M value around 0.5-1. Several candidate genes including “housekeeping” genes (rpsL), others involved in the general metabolism (gltA, gap-1, proC1, proC2), cell division (mreB, ftsZ) or signaling functions (ffH) were tested and finally, gltA and proC2 were selected as reference genes. For relative RT-PCR, experimental triplicates were performed, including always an internal calibrator in each plate, for data normalization. Samples without cDNA were used as negative controls. PCR reactions contained 12.5 μl. of iQ™ SYBR Green Superrnix (2x) (Bio-Rad, USA), 1 μl forward primer (10 μM), 1 μl reverse primer (10 μM), 2 μl of cDNA. (1/10 diluted), and was made with milliQ water up to 20 μl. The PCR cycling conditions were: 50° C./2 min and 95° C./10 min, followed by 40 cycles of 95° C. /15 s-60° C./30 s 72° C. /30 s, with a final extension at 72° C./10 min. Fluorescence was measured at the end of each cycle. For the melting curve, an initial denaturation step at 95° C./10 min was set up, followed for increments of 0.5° C./5 s starting with 65° C. up to 95° C., and continue signal acquisition. The relative expression ratio of the target genes was calculated automatically with the CFX software (Bio-Rad, USA) using the standard error of the mean and the normalized expression method (ΔΔ(Ct)). Values are expressed as Normalized fold increases in expression.

Culture Conditions for PHA Production

3-methylbenzoate (3-MB) was used as inducer for the activation of the XylS transcriptional activator by the Pm promotor that drives the T7 polymerase gene, which in turns, triggers the expression of the phaC2 synthase. In order to determine optimal conditions for phaC2 expression/PHA synthesis in PpU 10-33, concentrations of 3-MB (from 0.2-3 mM), times of induction (OD₅₅₀ₙₘ 0.4-1.5), and carbon sources concentrations were raised in different conditions. Erlenmeyer flasks (2 liter) containing 400 ml of MM modified medium (Martinez-Blanco et al, 1990) plus 0.1% of yeast extract, 15 mM sodium octanoate and appropriate antibiotics were inoculated with a cell suspension of an overnight culture at 30° C. on MM agar plates with 20 mM succinate. Flasks were then incubated at 30° C. in a rotary shaker (INFORS AG, Switzerland) at 180 rpm. Once the cultures reach an OD₅₅₀ₙₘ of about 0.8, the culture was split into two (1 liter Erlenmeyer flasks containing 200 ml) and 3-MB added to a final concentration of 0.5 mM to one of the flasks. At the same time a second pulse of sodium octanoate (20 mM) was added. For the wild type control strain, the procedure was the same but without the induction. Samples were collected every 24 h and the biomass (CDW, cellular dry weight), PHA, OD₅₅₀ₙₘ, Nile red staining and NH₄⁺ concentration determined. For CDW determination, samples were dried at 80° C. for 24 h and expressed in g/l of original culture.

PHA Extraction and Purification

Culture samples were centrifuged at 6,500 xg for 15 min at 4° C. (Allegra 25R, Beckman Coulter, USA), and pellets washed twice in distilled water and lyophilized (Lyophilizer alpha 1-4 LSC, Christ, Germany) at −59° C. and 0.140 mbar, Five ml samples were taken along the growth phase to monitor the PHA production and were lyophilized as described above. The lyophilized biomass was extracted with 10 ml chloroform for 3 h at 80° C. as described previously (Basas-Galia et al, 2012). PHA content (% wt) is defined as the percentage of CDW represented by PHA.

NMR Analysis

For ¹H-NMR analysis, 5-10 mg of polymer was dissolved into 0.7 ml of CDCl, and 5-10 mg of polymer was used for recording the ¹³C spectra. ¹H and ¹³C NMR spectra were recorded at 300K on a Bruker DPX-300 NMR Spectrometer locked to the deuterium resonance of the solvent, CDCl₃. Chemical shifts are given in ppm relative to the signal of the solvent (¹H: 7.26,¹³C 77.3) and coupling constants in Hz. Standard Bruker pulse programs were used throughout.

Detection of Molecular Weights of PHA

Average molecular weights were determined by gel permeation chromatography (GPC) in a HPLC system (Waters 2695 Alliance separations Module) with a column Styragel HR5E and equipped with a 2414 differential-refractive index detector (Waters, USA). Tetrahydrofuran (THF) was used as eluent at 45° C. and flow rate of 0.5 ml min⁻¹ (isocratic). Sample concentration and injection volume were 0.5 mg ml⁻¹ and 50 μl, respectively. The calibration curve was obtained using polystyrene standards kit (Fluke) in the Mw range of 10,000-700,000 g mol⁻¹.

Thermal properties of PHAs

The thermal properties of the microbial polyesters were determined by differential scanning calorimetry (DSC), using 10-20 mg of the purified polymer for analysis. DSC analyses were performed with a DSC-30 (Mettler Toledo Instruments, USA). Samples were placed on an aluminium pan and heated from −100° C. to 400° C. at 10° C. minⁱ under nitrogen (80 ml/mm), All data were acquired by STARe System acquisition and processing software (Mettler Toledo),

### Example 1: Hyper-Expression of phaC2 in Pseudomonas putida U

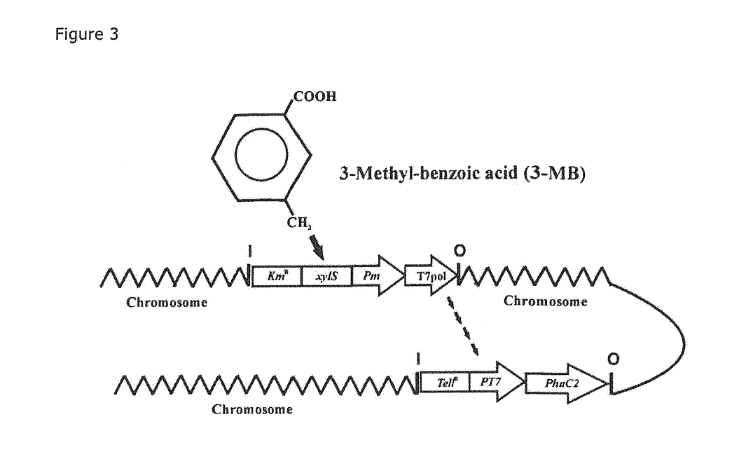

A bipartite, mini-transposon-based hyper-expression system for the PpU PhaC2 synthase, consisting of (i) a specialized mini-Tn5, pCNB1xylS/Pm:;77pol, expressing T7 polymerase from the Xy1S-3-metylbenzoate (3-MB)-regulated promoter Pm; and (ii) a hybrid pUT-miniTn5-Tel derivative expressing phaC2 from the T7 polymerase promoter was designed (see FIG. 3). The two minitransposon components were separately and randomly inserted into the P. putida U (in the following “PpU”) chromosome. The best PHA producer was selected after two rounds of screening, involving semi-quantification of PhaC2 production by SDS-PAGE separation of cellular proteins and inspection of PHA granule formation by fluorescence microscopy of Nile Red-stained cells. This strain was designated PpU 10-33.

In the following it will be referred to the non-induced cultures as NI and the cells induced with 0.5 mM of 3-MB as I. The effect of the phaC2 gene dosage in PHA content in the recombinant strain PpU 10-33 was assayed. Cultures were grown in modified MM with sodium octanoate given in two pulses of 15 mM and 20 mM (the second pulse was given in the moment of the induction), respectively. The peak biomass production was reached after 48 h for both strains, PpU and PpU10-33 (3.1 and 3.2 g 1-1 CDW, respectively). The results are shown in Table 1:

TABLE 1
Biomass yields of strain PpU, PpU 10-33 and PpU 10-33-ΔphaZ

| | CDW (g 1⁻¹) | | | | |
|---|---|---|---|---|---|
| Time | | PpU 10-33 | PpU 10-33 | PpU 10-33- | PpU 10-33- |
| (h) | PpU | (NI) | (I) | ΔphaZ (NI) | ΔphaZ (I) |
| 24 | 1.31 | 1.36 | 1.09 | 1.49 | 1.20 |
| 48 | 3.07 | 2.52 | 3.16 | 1.83 | 3.10 |
| 72 | 2.50 | 2.42 | 2.39 | 3.11 | 3.29 |
| 96 | 2.13 | 2.16 | 2.68 | 3.20 | 3.25 |

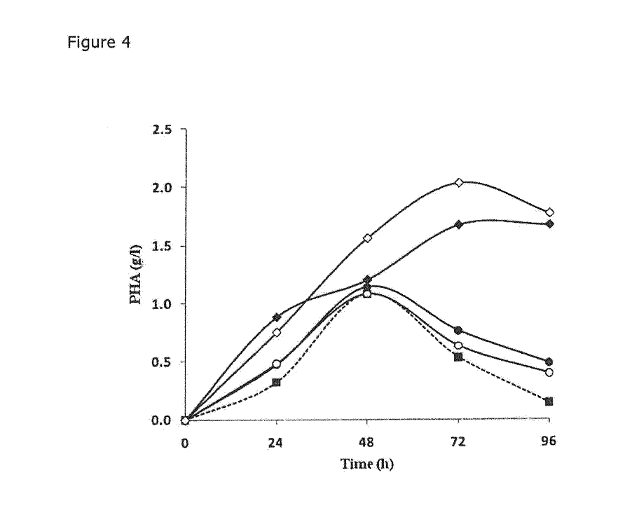

Cells exposed to 3-MB were able to accumulate higher amounts of PHA (44%) during the first 24 hours of culture, compared with the wild type and non induced cells (24.4% and 34.6%). The results are shown in the following Table 2 and FIG. 4:

TABLE 2
PHA yields in PpU, PpU 10-33 and PpU 10-33-ΔphaZ uninduced (NI) and
induced (I)

| | ^{a}PHA (g 1⁻¹) | | | | | ^{b}PHA (% wt) | | | | |
|---|---|---|---|---|---|---|---|---|---|---|
| | | | | PpU | PpU | | | | PpU | PpU |
| | | PpU | PpU | 10-33 | 10-33 | | PpU | PpU | 10-33 | 10-33 |
| Time | | 10-33 | 10-33 | ΔphaZ | ΔphaZ | | 10-33 | 10-33 | ΔphaZ | ΔphaZ |
| (h) | PpU | (NI) | (I) | (NI) | (I) | PPU | (NI) | (I) | (NI) | (I) |
| 24 | 0.32 | 0.47 | 0.48 | 0.88 | 0.75 | 24.4 | 34.6 | 44.0 | 59.1 | 62.5 |
| 48 | 1.08 | 1.14 | 1.08 | 1.20 | 1.56 | 35.2 | 45.2 | 34.2 | 65.6 | 50.3 |
| 72 | 0.53 | 0.76 | 0.63 | 1.67 | 2.03 | 21.2 | 31.4 | 26.5 | 53.7 | 61.7 |
| 96 | 0.14 | 0.48 | 0.39 | 1.67 | 1.80 | 6.6 | 20.5 | 14.6 | 52.2 | 54.5 |

Cultures were grown in modified MM with sodium octanoate 35 mM (given in two pulses of 15 and 20 mM) and were induced (I) with 0.5 mM 3-MB at an OD₅₅₀ₙₘ of 0.8 or not induced (NI).

PHA levels in the hyperexpressing strain were around 50% higher than those in the parental strain at 24 h but were around 25% lower than those of the parental strain at 48 h and similar at 72 h, suggesting that an increase in PhaC2 causes a transient increase in PHA, which in turn provokes an increase in depolyrnerization activity until levels are normalized. Importantly, the PHA percentage of cellular dry weight (% wt) dropped precipitously after 48 h from 35% to 7% wt, in the case of PpU, and from 39% to 15% wt, in the case of PpU 10-33 induced cultures.

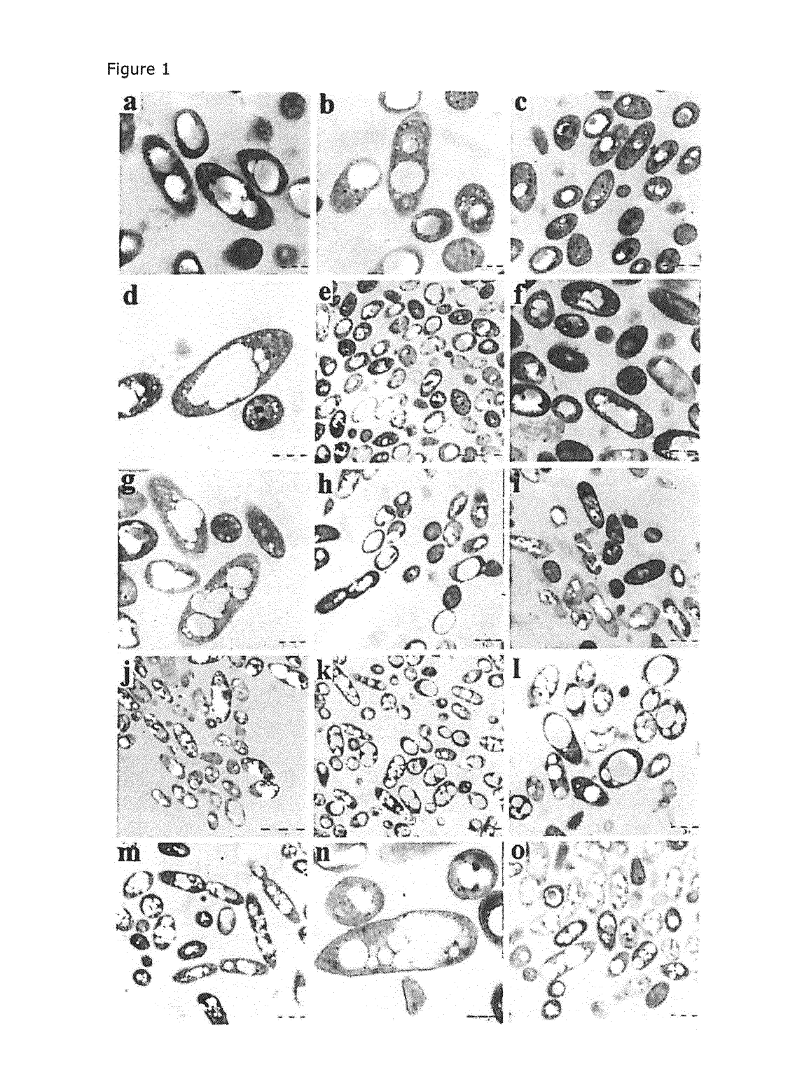

The reason why non-induced cultures of Poll 10-33 also showed a 50% increase in PHA accumulation over that of the wild-type strain at 24 h was not investigated further, but was assumed to reflect leakiness of the 17 promoter (also indicated by RT-PCR results), The highest biomass levels, 3.07 g in the case of PpU, and 2.67 g l⁻¹ (uninduced, NI) and 2.73 g l⁻¹ (induced, I) in the case of Poll 10-33 (FIG. 1A, Table 1), and PHA accumulation, 1.08 g l⁻¹, 0.74 g l⁻¹ and 1.07 g respectively (FIG. 4, Table 2), were attained at 48 h of cultivation with both strains. After 48 h, biomass and PHA levels dropped, with PHA levels diminishing or falling more significantly than biomass levels. The PpU 10-33 strain gave higher yields of PHA, expressed as percentage of biomass, at almost all sampling times. The highest PHA yield measured in this experiment, 44% wt, was obtained in PpU 10-33 induced cells at 24 h, compared to 24% wt in PpU and 35% wt in uninduced PpU 10-33 cells (Table 2). At 48 h, when the highest biomass yield was obtained, the highest absolute yield, 41% of cellular dry weight (COW) of PHA, was obtained in uninduced cells of 10-33, compared with 35% wt in PpU and 40% wt in induced PpU 10-33 cultures. Thus, the effect of induction is seen primarily in relatively young cultures. Importantly, the percentage of PHA dropped precipitously after 48 h to 7% wt in the case of PpU and 15-22% wt in the case of PpU 10-33.

### Example 2: Effect of the ΔphaZ Mutation on PHA Production

A phaZ deletion mutant of the PpU 10-33 strain, designated PpU 10-33-ΔphaZ, was created and subsequently assessed for PHA accumulation. As can be seen in FIG. 4 and Table 2, cultures of the mutant exhibited higher PHA levels (62% wt) and, in contrast to the situation with the PhaZ-producing strains, these levels were maintained until at least 96 h of cultivation. Thus, the ΔphaZ knockout phenotype suggests that the PhaZ depolymerase is a major determinant of PHA accumulation and maintenance in the cell.

### Reference Example: Complementation of the ΔphaZ-PpU10-33 Mutant

In order to causally relate the ,ohaZ gene mutation to the observed phenotype, and to rule out any indirect effects on expression of the pha cluster, the phaZ gene was PCRamplified, cloned in the pBBR1MCS-5 plasmid vector, and introduced into the PpU 10-33-ΔphaZ strain. PHA production and maintenance in the complemented mutant, PpU 10-33-ΔphaZ pMC-phaZ, designated strain pMC-phaZ was then assessed. Table 3 shows the biomass and PHA yields of the PpU 10-33 strain, its phaZ deletion mutant and the complemented derivative, after growth for 44 h in modified MM with sodium octanoate (20 mM).

TABLE 3
Effect on PHA yields of accumulation. PhaZ constructions and comple-
mentation of the defect.

| | ^{a} CDW | ^{b} PHA | ^{c} PHA |
|---|---|---|---|
| Strains | (g 1⁻¹) | (g 1⁻¹) | (% wt) |
| PpU 10-33 (NI) | 2.11 | 0.45 | 21.0 |
| ΔphaZ-PpU10-33 (NI) | 2.18 | 0.90 | 41.0 |
| pMC-PhaZ (NI) | 1.98 | 0.10 | 5.0 |

Biomass yields for the three stains were similar at about 2 g l⁻¹ whereas PHA yields were 21% wt for the PpU 10-33 strain, 41% wt for its ΔphaZ mutant, and 5% wt for the complemented strain. The lower than wild-type levels of PHA in the complemented strain presumably reflects higher cellular depolymerase levels, resulting from the complementing gene being located on a multicopy vector.

Polymer Characteristics

Since hyperexpression of PhaC2 polymerase and inactivation of PhaZ depolymerase may entrain changes in the normal cellular stoichiometry and activity of PHA proteins, and associated proteins, other changes in phenotypes may result from these genetic manipulations. To assess this possibility, the ultrastructure of the PHA granules in cells of the different constructs was compared by transmission electron microscopy (TEM). FIG. 1 shows that the PpU wild-type strain (FIG. 1A-C) contains one or two defined PHA granules per cell, distributed evenly within the cytoplasm, while the PpU 10-33 phaC2 hyperexpression strain (FIG. 1D-F) tends to contain one main granule with a morphology suggestive of the coalescence of smaller granules. This is particularly evident in the induced cultures, specifically during the mid-exponential growth phase. The phaZ deletion mutant tended to have multiple granules, some of which had irregular boundaries suggestive of granule fusion (FIG. 1G-I). The microscopic analysis also confirmed the results shown in FIG. 4, namely that intracellular PHA accumulated in the PpU and PpU 10-33 strains starts to diminish after 48 h of cultivation, whereas the mutant lacking the depolymerase maintained accumulated PHA until the end of the experiment.

Given that the two PHA syntheses of PpU have slightly different substrate specificities, with PhaC2 exhibiting a preference for 3-hydroxyhexanoyl-CoA and PhaC1 biased towards 3-hydroxyoctanoyl-CoA (Arias et al., 2008), it was possible that hyperexpression of the PhaC2 polymerase in PpU 10-33 might alter the monomer composition and/or physicochemical properties of the polymer produced. Table 4 shows that PHAs produced during growth on sodium octanoate by PpU, PpU 10-33 and its phaZ deletion mutant had similar compositions, as determined by NMR, and were copolymers of P(3-hydroxyoctanoate-co-3-hydroxyhexarioate), composed of 3-hydroxyoctanoate (91.4-92.5% mol) and 3-hydroxyhexanoate (7.58.6% mol).

TABLE 4
physico-chemical properties of the PHA from different strains

| | | | | | | | Monomer | |
|---|---|---|---|---|---|---|---|---|
| | ^{a} Mn | ^{b} Mw | | ^{d} Tg | ^{e} Tm | ^{f} Td | composition (% mol) | |
| Strains | (kDa) | (kDa) | ^{c} PI | (° C.) | (° C.) | (° C.) | 3-HHx | 3-HO |
| PpU | 76.6 | 126.3 | 1.65 | −35.90 | 61.40 | 294..03 | 8.6 | 91.4 |
| PpU 10-33 NI | 75.7 | 132.9 | 1.76 | −35.92 | 59.68 | 294.93 | 7.5 | 92.5 |
| PpU10-3 I | 74.9 | 141.1 | 1.88 | −37.16 | 59.21 | 294.04 | 8.4 | 91.6 |
| PpU10-33 ΔphaZ NI | 52.1 | 95.6 | 1.83 | −40.82 | 59.60 | 293.84 | 8.6 | 91.4 |
| PpU10-33 ΔphaZ I | 50.1 | 96.2 | 1.92 | −36.09 | 61.57 | 293.65 | 8.7 | 91.3 |

Polymers were obtained from PpU, PpU 10-33 and PpU 10-33-ΔphaZ uninduced (NI) and induced (I) cells cultured in modified MM octanoate 35 mM (given in two pulses of 15 mM and 20 mM)
^{a} number average molecular weight;
^{b} weight-average molecular weignt;
^{c} polydispersity index (Mw/Mn);
^{d} melting temperature;
^{e} enthalpy of fusion;
^{f} decomposition temperature; 3-HHx = 3-Hydroxyhexanoate; 3-HO = 3-hydroxyoctanoate

Also, the glass transition temperature of the three polymers, Tg −35.9 to −40.8° C. (Table 4), was in agreement with the Tg described previously for medium chain length (mcl)-PHA5, and they had similar melting temperatures (Tm, 59-61° C.), indicating similar crystallinity grades.

However, the polymers differed in length: the molecular weights (Mw and Mn values) of the polymers from the PpU parental strain and the PpU 10-33 (PhaC2 polymerase hyperexpressing construct) were similar, ranging from 126-142 and 74-77 kDa respectively, whereas those from the PhaZ knockout were considerably lower, 96 and 50 kDa respectively

Transcriptional Analysis of the pha Operon by Relative RT-PCR in PpU, PpU10-33 and PpU10-33-ΔphaZ

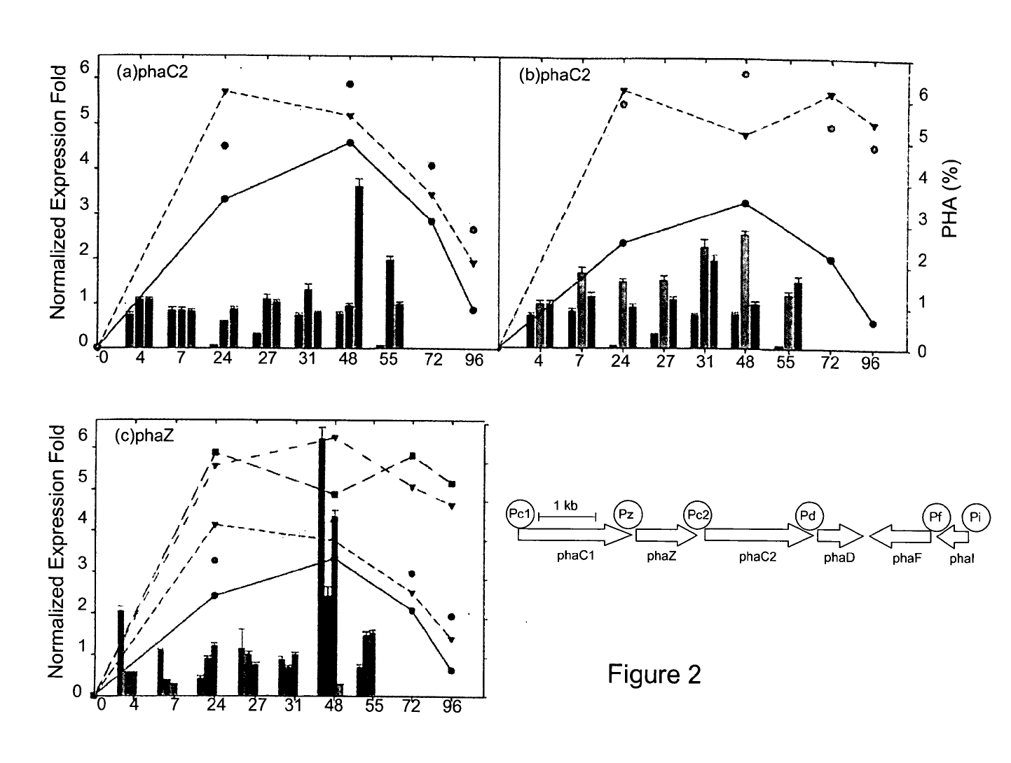

In order to investigate the relationship between PHA turnover and the hyperexpression of phaC2 and phaZ inactivation, transcriptional analysis was carried out by relative RT-PCR of the pica cluster (FIG. 2) in the three strains. Reference genes for the RT-PCR data normalization were gltA and proC2

In the wild type, no major changes were detected in transcript levels of the two PHA polymerases, PhaC1 and PhaC2, during the first 24 h of cultivation (P>0.1), and this was accompanied by a steady increase in PHA accumulation. However, a twofold increase (P<0.001) in phaZ transcripts was measured at 4 h, corresponding to the onset of PHA production, which then fell back to lower levels. At 48 h, correlating with maximum levels of PHA accumulation, a rapid and substantive increase in the transcription of phaC1 was observed (4.5-fold, P<0.0001) and, in parallel, a sixfold increase (P<0.001) in phaZ transcriptional activity. This was followed by a rapid decrease in the PHA content (FIG. 2), and phaC1 and phaZ transcript levels. These results are indicative of a finely tuned coupling of phaC1 transcription and PHA accumulation, on one hand, and phaZ transcription and PHA mobilization, on the other.

In the case of the PpU 10-33 strain, expression of the phaC2 gene was, as expected, found to be higher than in the PpU parental strain throughout the cultivation period (P<0.008) and especially at 48 h, when it peaked (3.5-fold increase, P<0.0001). Interestingly, the expression of phaC1 in this strain was mostly lower than in PpU, especially in induced cultures at 7 h, 24 h and 48 h, suggesting that hyperexpression of phaC2 negatively Influences expression of phaC1 (FIG. 2). However, even though hyperexpression of phaC2 resulted in decreasing expression of phaC1, the combined cellular synthase activity resulted in an increased PHA production. Transcription levels of phaZ in PpU 10-33 tended to be similar to those in the parental strain, except at 24 h, when it was higher, correlating with the higher expression of phaC2 and in cultures older than 48 h in which it was also higher, consistent with the higher levels of PhaC2 and PHA. There is thus also a strong coupling of PhaC2 polymerase and depolymerase synthesis. In the PpU 10-33-DphaZ strain, significantly higher transcription levels of phaC2 were observed throughout the cultivation period when compared with the wild type (P 0.0005-0.017), which is consistent with the higher PHA yields obtained (from 60% wt to 66% wt, see FIG. 4). In the case of phaC1 also higher levels were measured at 24 and 38 h, but only when phaC2 was induced (P<0.0017). Thus, inactivation of phaZ not only prevents turnover and recycling of synthesized PHA, but also allows higher transcription levels of the PHA polymerases.

Solvent Extraction Methods for PHA Recovery from PpU Strains

The extraction conditions for the PHA produced in the modified PpU strains were investigated in different solvent systems, selected from chloroform, dichloromethane and acetone. Extractions were performed at two different temperatures, room temperature (RT) and 80° C., and using three times of extraction (30 min, 1 h, 3 h and 18 h). The lyophilized cells used in this experiment were obtained following the standard culture conditions for P. putida U and its derivatives: the three strains were cultivated in MM+0.1% YE for 72 h, at 30° C. and 200 rpm, in 1 L flask containing 200 ml of medium and using octanoic acid (10+20 mM) as substrate. The mutant strains (PpU 10-33 and the PpU 10-33-Δpha2) were not induced. Samples of 40 mg of lyophilized biomass were disposed in the extraction tubes, resuspended in the corresponding solvent and extracted under the different conditions described above, Percentages of PHA recovery are referred to the initial 40 mg of lyophilized biomass (Table 5), The classical extraction with chloroform (3 h and 80° C.) was used as control.

TABLE 5
PHA recovery (% wt) using different solvents, time of extraction and
temperatures.

| | | 3 h-80° C. | 1 h-RT | 3 h-RT | 18 h-RT |
|---|---|---|---|---|---|
| PpU | CHCl₃ | 33.1 ± 0.9 | 30.6 ± 0.1 | 32.4 ± 2.3 | 30.6 ± 4.7 |
| | CH₂Cl₂ | 34.4 ± 2.0 | 31.5 ± 0.7 | 30.7 ± 0.6 | 31.6 ± 2.5 |
| | Acetone | | | 21.3 ± 1.5 | 25.1 ± 0.5 |
| PpU 10-33 | CHCl₃ | 36.4 ± 0.8 | 33.6 ± 1.2 | 34.0 ± 1.1 | 33.2 ± 1.7 |
| | CH₂Cl₂ | 30.0 ± 2.8 | 34.3 ± 3.2 | 34.1 ± 1.9 | 34.4 ± 2.3 |
| | Acetone | | | 26.8 ± 2.5 | 27.9 ± 1.7 |
| PpU 10- | CHCl₃ | 58.8 ± 3.2 | 58.2 ± 0.2 | 58.0 ± 0.2 | 56.9 ± 2.3 |
| 33ΔphaZ | CH₂Cl₂ | 59.5 ± 1.2 | 58.7 ± 4.3 | 56.6 ± 2.6 | 58.3 ± 0.1 |
| | Acetone | | | 57.3 ± 1.1 | 57.4 ± 2.2 |

Results are means of triplicates ± standard deviation. CH₂C1₂: dichloromethane and CHCl₃: chloroform

In PpU 10-33-ΔphaZ, no significant differences among the conditions were observed and the percentage of PHA recovery ranged between 56 and 59% wt. However, in the PpU (wild type) and the single mutant, the percentages of PHA recovery, when acetone was used as solvent, were between 21-28% wt, while for the other solvents, the percentages of recovery were about 31-34% wt.

Assuming that for the control conditions (chloroform, 3 h and 80° C.) the PHA recovery was the maximum (100%), a relative percentage of PHA recovery was calculated in order to evaluate whether there was any difference among the strains. In case of chloroform as the extraction solvent, no significant differences were observed in any of the strains. Nevertheless, the relative percentage of PHA recovery was slightly higher in the ΔphaZ mutant (96-98 rel. %), while for the wild type and the single mutant the recovery was at about 91-93 rel. %.

Similar behaviour was observed when dichloromethane was used as solvent. The ΔphaZ mutant showed rel. % PHA recovery of 96-100 rel. %, while the two other strains (revealed values of PHA recovery between 93-96 rel. %.

The most significant differences could be observed, when acetone was used as solvent. Among the solvents tested, acetone is the most environmentally friendly one, but at the same time probably also the solvent with the least extraction capacity. This latter aspect likely was key to unravel the differences in the percentages of PHA recovery between the double mutant (PpU 10-33-ΔphaZ) and the two other strains (PpU and PpU 10-33).

The ΔphaZ mutant is the one, which showed the highest yield of recovery, 97-98 rel. %. Surprisingly no differences were observed after 3 h or 18 h of extraction, indicating that 3 h of extraction is already sufficient. In contrast, in the other two strains (PpU and PpU 10-33), the relative percentages of PHA recovery decreased drastically being 64 rel. % and 74 rel. %, respectively, after 3 h of extraction. These percentages increased to some extent after 18 h of extraction, up to 76 rel. % and 78 rel % for the wild type and the single mutant, respectively.

Remarkable are the results obtained with acetone as solvent and short time of extraction (30 min) that showed the highest differences in the relative PHA recovery percentages, being of 50-55 rel. % for the wild type (PpU) and the single mutant (PpU 10-33) and 86 rel. % in the double mutant (PpU 10-33-ΔphaZ). Thus, acetone is the solvent in which the strains displayed the most pronounced differences, with the double mutant (PpU 10-33-ΔphaZ) being the strain that exhibited the highest yield of relative PHA recovery.

Thus, for the strain PpU 10-33-ΔphaZ acetone represents an equally good and environmentally friendly alternative solvent to replace chloroform in the PHA recovery process. Furthermore, the results indicate that is effect is largely facilitated by the cell morphology i.e. PHA granula coalescence.

Optimization of substrate dependant PHA production of PpU 10-33-ΔphaZ

The engineered strain was initially cultivated in three different media (E2, MM+0.1% YE and C-Y(2N)) and eight different substrates were tested (hexanoate (C6), heptanoate (C7), octanoate (C8), decanoate (C10), 10-undecenoate

(C11:1), oleic acid, linoleic acid and glycerol). The media had the following compositions:

1. E2 medium as described by Vogel 81. Borner (1956, 3, Biol. Chem. 218: 97-106). 2. MM medium+0.1% yeast extract as described by Martinez-Blanko et al. (1990, 3. Biol. Chem, 265: 7084-7090).

3. C-Y medium as described by Choi et al. (1994, Appl. Environ. Microbiol. 60: 3245-3254) with regular or twice (C-Y(2N)) the nitrogen concentration (0.66 and 1.32 g/l (NH₄)₂SO₄).

The best results were obtained in MM+0.1% YE and C-Y(2N) media, thus kinetic production studies were carried out in these two media using the eight substrates and using P. putida U wild type (PpU) as control. Samples were taken every 24 h in all strain/medium/substrate combinations to determine biomass and PHA production. The best production yields regarding PHA production in the different culture conditions tested as well as the harvesting time are compiled in Table 6.

TABLE 6
Biomass and PHA production yields obtained with P. putida U (PpU)
and the engineered strain PpU 10-33-ΔphaZ cultivated in two
different media, MM + 0.1% YE and C-Y (2N)

| | PpU | | | | PpU 10-33-ΔphaZ | | | |
|---|---|---|---|---|---|---|---|---|
| | time | CDW | PHA | PHA | time | CDW | PHA | PHA |
| | (h) | (g/L) | (g/L) | (% wt) | (h) | (g/L) | (g/L) | (% wt) |
| MM + 0.1% YE | | | | | | | | |
| substrate | | | | | | | | |
| C6 (10 + 20 mM) | 72 | 1.69 | 0.04 | 2.4 | 72 | 1.65 | 0.15 | 9.1 |
| C7 (10 + 20 mM) | 72 | 1.38 | 0.23 | 16.7 | 72 | 2.04 | 0.67 | 32.8 |
| C8 (10 + 20 mM) | 48 | 2.56 | 1.05 | 41.0 | 48 | 3.25 | 1.82 | 56.0 |
| C10 (10 + 20 mM) | 72 | 3.40 | 1.14 | 33.5 | 72 | 2.49 | 1.21 | 48.6 |
| C11:1 (27 mM) | 72 | 0.46 | 0.26 | 56.5 | 72 | 0.42 | 0.23 | 54.8 |
| glycerol (3%) | 96 | 6.68 | 1.00 | 15.0 | 96 | 6.44 | 1.35 | 21.0 |
| glycerol (4%) | 120 | 6.09 | 0.78 | 12.8 | 120 | 6.31 | 1.44 | 22.8 |
| oleic (1%) | 96 | 5.90 | 2.09 | 35.4 | 96 | 5.73 | 2.33 | 40.7 |
| linoleic (1%) | 72 | 4.75 | 1.28 | 26.9 | 72 | 5.78 | 2.47 | 42.7 |
| C-Y (2 N) | | | | | | | | |
| substrate | | | | | | | | |
| C6 (10 + 20 mM) | 72 | 0.69 | 0.11 | 15.9 | 72 | 0.15 | 0.07 | 46.6 |
| C7 (10 + 20 mM) | 72 | 2.19 | 0.57 | 26.0 | 72 | 1.53 | 0.74 | 48.4 |
| C8 (10 + 20 mM) | 24 | 1.91 | 0.91 | 47.6 | 48 | 3.37 | 1.86 | 55.2 |
| C10 (10 + 20 mM) | 24 | 2.83 | 1.27 | 44.9 | 24 | 4.68 | 2.48 | 53.0 |
| C11:1 (27 mM) | 96 | 3.75 | 0.94 | 25.1 | 96 | 3.83 | 1.68 | 43.8 |
| glycerol (3%) | 120 | 3.97 | 0.31 | 7.8 | 120 | 4.09 | 0.64 | 21.0 |
| glycerol (4%) | 120 | 4.94 | 0.55 | 11.1 | 120 | 6.31 | 1.18 | 23.0 |
| oleic (1%) | 72 | 5.18 | 1.48 | 28.6 | 96 | 4.82 | 1.99 | 41.2 |
| linoleic (1%) | 96 | 5.68 | 1.72 | 30.3 | 96 | 4.21 | 1.51 | 35.7 |

C6: hexanoate; C7: heptanoate; C8: octanoate; C10: decanoate; C11:1: 10-undecenoate.

In most of the substrates tested, the PHA production was higher in the engineered strain than in the wild type, obtaining an increment that ranges from 6% to 300%. PpU-10-33-ΔphaZ2 showed a poor polymer production when cultivated in both media with hexanoate or 10-undecenoate as carbon source. In contrast, a significant increase in PHA production was observed when PpU 10-33-ΔphaZ was grown in C-Y(2N) using decanciate as substrate, with a PHA yield largely the PHA-yield obtained in the MM+0.1% YE with the same carbon source. The double mutant was able to accumulate up to 2.48 g/L. (53.0% wt) of PHA in 24 h when was cultured in C-Y (2N), while in MM+0.1% YE it took up to 72 h to produce 1.21 (48.6% wt) of PHA. In contrast, similar production levels were obtained when PpU-10-33-ΔphaZ was cultivated using octanoate, reaching a PHA production of 1.82-1.86 g/L (55.0-56.0% wt) in both media.

In general, PHA peak production in glycerol, oleic and linoleic acid required longer time of cultivation. In case of glycerol, PHA accumulation of the mutant was higher than for the wild type (21-23% wt vs. 8-15% wt, respectively). A similar pattern was observed with oleic acid and (partially) linoleic acid, although both latter substrates generally allowed for higher percentages of PHA accumulation (35-42% wt), even though there was a significant increase with respect the wild type (8-15% wt), the PHA production was lower in comparison with the other substrate tested.

The strain PpU-10-33-ΔphaZ showed the highest PHA yields when cultivated in MM+0.1% YE/octanoate, MM+0.1% YE/oleic acid and C-Y (2N)/decannate. Any of these three medium/substrate combinations are good candidates to scale up to small-scale (5L) bench-top bioreactors in order to enhance the PHA production.

Investigation of PHA-Production in the Absence of Antibiotic Pressure

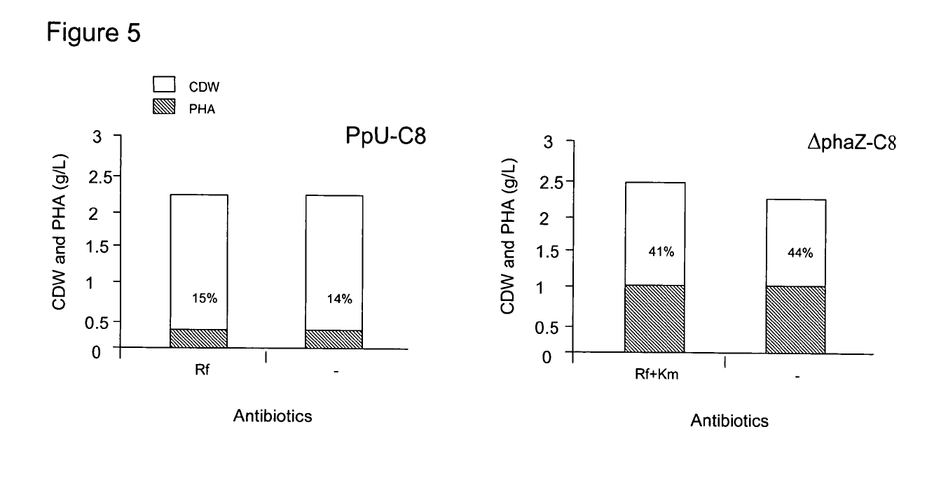

In order to facilitate the scale up of the process and to reduce the cost of the fermentation, the maintenance of the mutant strain under antibiotic pressure was studied. The engineered strain was usually preserved under Rifampicin (Rf), Kanamycin (Km) and Tellurite (Tell). The presence of Tellurite (Tell) and its oxidation in the culture provokes the darkening of the liquid media affecting the biomass measurements and recovery. In the following investigations the antibiotic was thus omitted from the cultures. Cultures with and without Tellurite were performed to evaluate its effect on the production yields. The investigations showed that no variations could be detected. Furthermore, in order to study the influence of the presence of Rifampicin and Kanamycin in the biomass and polymer production, the wild type and the engineered strains were cultured in mineral medium MM+0.1% YE using octanoate as substrate with and without the respective antibiotics Rifampicin (Rf) for the wild type and the combination Rifampicin+Kanamycin (Rf+Km) for the engineered strains. The results of these investigations are shown in FIG. 5.

No differences were observed in the biomass and polymer production, meaning that the presence or not of the antibiotics is not affecting to the production yields. Additionally, it was corroborated that the genotype of the engineered strains was not modified by the absence of the antibiotics. Both strains were cultured as previously described without antibiotic. At 48 h and 72 h, a dilution of each culture was plated in a LB plate without antibiotic and after 24h of incubation at 30° C., 50 colonies were picked and streaked on a LB plate+antibiotic and incubated for 24 h at 30° C. to verify the maintenance of the resistance pattern in each strain. After incubation, all the colonies grew in the plates with antibiotics, indicating that the absence of the antibiotics was not affecting the resistance phenotype, thus the resistance genotype should be preserved in the engineered strain.

The obtained results indicate that the cultivation of the double mutant, PpU 10-33-ΔphaZ, without the antibiotic (Rf+km) pressure and Tellurite is not affecting the PHA production.

### REFERENCES

Arias S., Sandoval, A., Arcos, M., Canedo, M L, Naharro, G., and Luengo, J. M. (2008) Micro Biotech 1: 170-176.

Basses, M. (2010) Isolation and Analysis of Storage Compounds. In Handbook of Hydrocarbon and Lipid Microbiology: Experimental Protocols and Appendices. Timmis K. N. (ed.). Berlin: . Springer Verlag, pp. 3725-3741.

Castaneda, M., Guzman, J., Moreno, S., and Espin, G. (2000) J Bacteriol 182: 2624-2628.

ChoiJ, Lee, S. Y. (1997) Bioprocess Eng. 17: 335-342.

Conte, E., Cetera, V., Greco, S., Russo, M., Alicata, R., Strano, L., Lombardo, A., Di Silvestro, S., and Cetera, A. (2006) App Microbiol Biotechnol 72: 1054-1062.

de Eugenio, L. I., Escapa, I. F., Morales, V., Dinjaski, N., Galan, B., Garcia, J. L., Prieto, M. A. (2010a). Environ Microbial 12: 207-221.

de Eugenio, L. I., Galan, B., Escapa, I. F., Maestro, B., Sanz, J. M. Garcia, J. L., Prieto, M. A. (2010b) Environ Microbial 12: 1591-1603.

Diederich, L., Roth, A., and Messer, W. (1994) Bio Techniques 16: 916-923.

Donnenberg, M. S., and Kaper, J. B. (1991) Infect Inmun 59: 4310-4317.

Galan, B., Dinjaski, N., Maestro, B., de Eugenio, L. I., Escapa, I. F., Sanz, J. M., Garcia, J. L., Prieto, M. A. (2011) Mol Microbiol 79:402-418.

Grage, K., Jahns, A. C., Parlane, N., Palanisamy, R., Rasiah, I. A., Atwood, J. A., Rehm, B. H. (2009) Biomacromolecules 10: 660-669.

Harayama, S., Rekik, M., Wubbolts, M., Rose, K. R., Leppik, R. A. and Timmis, K. N. (1989). J Bacterial 171 :5048-5055.

Herrero, M., de Lorenzo, V., and Timmis, K. N. (1990) J Bacteriol 172: 6557-6567.

Herrero, M., de Lorenzo, V., Ensley, B., and Timmis, K. N. (1993) Gene 134:103-106.

Hoffmann, N., and Rehm, B. H. (2005) Biotechnol Lett 27: 279-282.

Hrabak, O. (1992) FEMS Microbiol. Rev. 103: 251-256.

Huisman, G. W., Wonink, E, Melina, R., Kazemier, B., Terpstra, P., and Witholt, B. (1991) J Biol Chem 266: 2191-2198.

Kessler, B., and Witholt, B. (2001) J Biotechnol 86: 97-104.

Kraak, M. N., Kessler, B., and Witholt, B. (1997) Eur J Biochem 250: 432-439.

Luengo, J. M., Garcia, B., Sandoval, A., Naharro, G., Olivera, E. R. (2003) Curr Opin Microbial 6: 251-260.

Madison, L. L., and Huisman, G. W. (1999) Microbiol Mol Biol Rev 63: 21-53.

Martinez-Blanco, H., Reglero, A, Rodriguez-Aparicio, L. B., and Luengo, J. M. (1990). J Biol Chem. 265: 7084-7090.

Prieto, M. A., Bühler, B., Jung, K., Witholt, B., and Kessler, B. (1999a) J Bacteriol 181: 858-868.

Prieto, M. A., Kellerhals, M. B., Bozzato, G. B., Radnovic, D., Witholt, B., and Kessler, B. (1999b) Appl Environ Microbial 65: 3265-3271.

Quant, J., and Hynes, M. P. (1983) Gene 127: 15-21.

Ren, Q., de Roo, G., Ruth, K., Witholt, B., Zinn, M., Thöny-Meyer, L. (2009a) Biomacromolecules 10: 916-22.

Ren, Q., de Roo, G., Witholt, B., Zinn, M., and Thöny-meyer, L. (2010) BMC Microbiol 10: 254.

Sambrook, J., and Russell, D. W. (2001) Molecular cloning. A laboratory manual. Cold Spring Harbor, N.Y.: CSHL Press.

Sanchez-Romero, J. M., Diaz-Orejas, R., and de Lorenzo, V. (1998) Appl Environ Microblol 64: 4040-4046.

Selyaraj, J., and lyer, V. N. (1983) J Bacteriol 156: 1292-1300.

Solaiman, D. K., Ashby, R. D., and Foglia, T. A. (2003) Appl Microbial Biotechnol 62: 536-543.

Spurr, A. R. (1969) J. Ultrastruct Res 26: 31-43.

Steinbüchel, A. Polyhydroxyalkanoic adds in Biomaterials, D. Byrom, ed., MacMillan Publishers, Basingstoke (1991), p. 123 ff.

Steinbüchel, A., Aerts, K., Babel, W., Follner, C., Liebergesell, M., Madkour, M. H., et al, (1995) Can J Microbial 41: 94-105.

Uchino, K., Saito, T., Gebauer B., Jendrossek, D. (2007) J Bacteriol 189: 8250-8256.

Williams and Peoples (1996) Chemtech 26, 38-44.

York, G. M., Stubbe, J., and Sinskey, A. J. (2002) J Bacteriol 184: 59-66.

Annex 1: Strains, mutants and plasmids used

| Vectors and | | |
|---|---|---|
| constructions | Description | Reference |
| RK600 | Cm^{R}, oriColE1, oriV, RK2mob^{+tra+}. Helper plasmid in triparental | Herrero et al., |
| | conjugation events. | 1990 |
| pUC18Not/T7 | Ap^{R}, oriColE1, lacZα+, promoter lac, pUC18NotI derivative | Herrero et al., |
| | vector in which a synthetic T7 promoter sequence has been | 1993 |
| | introduced from the EcoRI site of the polylinker. | |
| pCNB1mini-Tn5 | Km^{R}, tnp⁻, xylSPm promoter, T7 RNA polymerase. | Harayama et al, |
| xylS/Pm::T7pol | | 1989; Herrero et |
| | | al., 1993 |
| pUTminiTn5-Tel | Tel^{R}, tnp⁻. | Sanchez-Romero |
| | | et al., 1998 |
| pGEM ®-T Easy | Ap^{R}, oriColE1, lacZα+, SP6 T7, lac promoter.. | PROMEGA |
| pJQ200 (KS/SK) | GM^{R}, orip15A, Mob+, lacZα+, sacB, vector used for generate | Quandt & Hynes, |
| | deletions by double recombinant events. | 1993 |
| pBBR1MCS-5 | GM^{R}, orip15A, Mob+, lacZα+, promoter lac broad-host-range | Kovach et al., |
| | cloning and expression vector. | 1995 |
| pBBR1MCS-3-phaC2 | A pGEMT Easy insert from position −26 to +1832 from ATG of | Arias et al., 2008 |
| | phaCl was cloned into pBBR1MCS-3 vector using the restriction | |
| | sites SacII-SacI. Tc^{R}. | |
| pUC18Not/T7-phaC2 | pUCl8Not/T7 containing the phaC2 excised from the | This study |
| | pBBR1MCS-3-phaC2 construct and cloned using the restriction | |
| | site EcoRI. | |
| pUTminiTn5-TEl- | Mini-Tn5-Tel containing the T7promoter-phaC2-excised as a NotI | This study |
| T7phaC2 | cassette from pUC18Not/T7-phaC2. | |
| pMS-phaC1C2- | pMS vector containing a synthetic DNA cassette (3531 bp) | This study |
| 0941347 | encoding the PhaC1 and PhaC2 synthases, and cloned into the | (GENEART AG) |
| | HindIII and KpnI restriction sites. SM^{R} | |
| pJQ200SK-phaC1C2 | A synthetic DNA insert from position −106 to +3383 from ATG of | This study |
| | phaC1 cloned into the pJQ200SK by using the restriction site NotI. | |
| pBBR1MCS-5-phaZ | A pGEMT Easy insert from position −27 to +890 from ATG of | This study |
| | phaZ cloned into the KpnI-XbaI sites of pBBR1MCS-5. | |
| Strains | | |
| E. coli DH10B | F-, mcrA, Δ(mrr-hsdRMS-mcrBC) Ø80dlacZ1M15, ΔlacX74, | Invitrogen |
| | deoR, recAl, endAl, araD139, Δ(ara, leu)7697, galU galK, λ-, | |
| | rpsL, nupG. | |
| E. coli CC18λpir | F-, Δ(ara, leu), araD, ΔlacX74, galE, galK, phoA20, thi-I rps-1, | Herrero et al., |
| | rpoB, argE(Amp), recA, thi pro hsdRM+, RP4-2-Tc (CC18 | 1990 |
| | lysogenised with the λpir phage) | |
| PpU | P. putida U strain (CECT4848), Rf^{R}. | Martinez-Blanco |
| PpU-pCNB1mini- | P. putida U containing pCNB1mini-Tn5xylS/PM::T7pol vector. | et al., 1990 |
| Tn5xylS/Pm::T7pol | Km^{R} Rf^{R} | This study |
| PpU 10-33 | P. putida U containing pCNB1mini-Tn5xylS/PM::T7pol and | This study |
| | pUTminiTn5-Tel-phaC2. Km^{R} Tel^{R} Rf^{R}. | |
| ΔphaZ-PpU 10-33 | PhaZ deleted PpU 10-33 Km^{R} Tel^{R} Rf^{R}. | This study |
| pMC-PhaZ | ΔphaZ PpU 10-33 complemented by the phaZ gene (pBBR1MCS- | This study |
| | 5-phaZ-1-ΔphaZ-PpU 10-33). Gm^{R} Km^{R} Tel^{R} Rf^{R} | |

ANNEX 2
List of oligonucleotides employed for the PT-PCR assay in this study.

| Gene | Forward Primer (5′3′) | Reverse Primer (5′3′) |
|---|---|---|
| ¹16s ribosomal DNA | ACGATCCGTAACTGGTCTGA | TTCGCACCTCAGTGTCAGTA |
| (16s rDNA) | | |
| ¹Citrate synthase (glpA) | GCCGATTTCATCCAGCATGGTC | TGGACCGGATCTTCATCCTCCA |
| PP_4194 | | |
| ¹Ribosomal protein S12 | GGCAACTATCAACCAGCTGGT | GCTGTGCTCTTGCAGGTTGTG |
| (rpsL) PP_0449 | | |
| ¹Glyceraldehyde 3-phosphate | CTTGAGGTTGACGGTGAGGTC | AGGTGCTGACTGACGTTTACCA |
| dehydrogenase (gap-1) | | |
| PP_1009 | | |
| ¹Signal recognition particle | CGGTAGTCAAGGATTTCGTCAAC | CACCATCACGCTCTTTTTCTTG |
| protein Ffh (ffH) PP_1461 | | |
| ¹Rod shape-determining | CGTGAAGTGTTCCTGATCGAAG | CCGATTTCCTGCTTGATACGTT |
| protein MreB (mreB) | | |
| PP_0933 | | |
| ¹Cell division protein FtsZ | CGGTATCTCCGACATCATCAAG | GAGTACTCACCCAGCGACAGGT |
| (ftsZ) PP_1342 | | |
| ¹Pyrroline-5-carboxylate | GCATTTACCAGCCCTTTGAAGC | CAATGACGAAAGGCAAATCGAC |
| reductase 1 (proC1) PP_3778 | | |
| ¹Pyrroline-5-carboxylate | CTCCCAACTGACCTTGCAGAC | GCTCCTTATTTGCCCAGTTGTTC |
| reductase 2 (proC2) PP_5095 | | |
| ²PHA synthase 1 (phaC1) | GCATGTGGCCCACTTTGGC | CCCGGTTCTTGCCCACTT |
| ²PHA depolymerase (phaZ) | AGCAGTTTGCCCACGACTACC | GGTGGATCTTGTGCAGCCAGT |
| ²PHA synthase 2 (phaC2) | GGCAACCCCAAGGCCTACTAC | CCGAGCGGTGGATAGGTACTG |
| ²Phasin PhaF (phaF) | GTCAGCTTCTCGATCTGCTTGGT | GAAGAAGACGGCTGAAGATGTAGC |
| ²Phasin PhaI (phaI) | CTCTTTGTCGATGCGTTTCTTG | CATGGCCAAAGTGATTGTGAAG |
| ²PhaD transcriptional | GAACGTATCCACCCTGGAGATT | ATAAGGTGCAGGAACAGCCAGTAG |
| regulator (phaD) | | |
| ²Long-chain-fatty-acid-CoA | CGTGATCAAGTACGTGAAGAAGATG | GTGAAGGCGTAGATGTGGTACAG |
| ligase 1 (fadD1) | | |
| ²Long-chain-fatty-acid-CoA | GCTGTACCACATCTATGCCTTCAC | GCCGGAGTTGGTGACTTTCAG |
| ligase 2 (fadD2) | | |

The numbers (^{1,2}) indicate whether the DNA from P. putida KT2440 or P. putida U was used as a template, respectively.

ANNEX 3
List of additional oligonucleotides used

| Primer | Sequence (5′3′) |
|---|---|
| M13F | GTAAAACGACGGCCAG |
| M13r | AGGAAACAGCTATGAC |
| PhaC1-check-F | GAATCGGTTGTGAAACTCATGCTC |
| PhaC2-check-R | CCTTGCCATGGAAGTGGTAGTACAG |
| RT-phaZ F_PpU | AGCAGTTTGCCCACGACTACC |
| RT-phaZ R_PpU | GGTGGATCTTGTGCAGCCAGT |
| phaZ-F-KpnI | GGGGTACCCCCACTTTTTCACGACAGAGTCGAACG |
| phaZ-R-XbaI | GCTCTAGAGCGCAACACTCCCTCGTCTTACC |

1. A genetically engineered form of a naturally PHA-producing microorganism, which has an increased number of copies compared to the wild type microorganism of at least one gene encoding a polyhydroxyalkanoate (PHA) synthase, wherein said increased number of copies provides a balanced overproduction of said PHA synthase and wherein the genetic engineering causes the microorganism to overproduce medium- or long-chain-length PHAs in an amount of at least 1.2 times compared to the wild type after 24 h, wherein the reference condition for assessing the overproduction is modified MM medium containing 15 mM sodium octanoate.

2. The genetically engineered microorganism of claim 1, wherein the gene encodes for the PhaC2 synthase or homologues thereof.

3. The genetically engineered microorganism of claim 1 or 2, wherein the expression of the PHA synthase is regulated by a promoter system, which is preferably protein based, more preferably a T7 polymerase/ T7 polymerase promoter system.

4. The genetically engineered microorganism of any one of claims 1 to 3, further having at least one modification in at least one gene encoding a protein involved in the degradation of PHA in said microorganism, wherein the modification causes complete or partial inactivation of the gene encoding a protein involved in the degradation of PHA, more preferably complete inactivation of said gene.

5. The genetically engineered microorganism of claim 4, wherein the protein involved in the degradation of PHA is a PHA depolymerase, preferably phaZ and homologues thereof.

6. The genetically engineered microorganism any one of claims 1 to 5, wherein the genetic modification is maintained in the microorganism on reproduction and/or cultivation, preferably both in the absence or presence of antibiotics.

7. The genetically engineered microorganism of any one of the preceding claims, wherein the genetic engineering causes the microorganism to overproduce medium chain polyhydroxyalkanoate(s) PHA, preferably in an amount of at least 1.5 times and more preferably at least 2 times compared to the wild type after 24 h, wherein the reference condition for assessing the overproduction is modified MM medium containing 15 mM sodium octanoate.

8. The genetically engineered microorganism of any one of the preceding claims, wherein the microorganism is selected from the group consisting of Pseudomonas putida, Pseudomonas aeruginosa, Pseudomonas syringae, Pseudomonas fluorescens, Pseudomonas acitophila, Pseudomonas olevarans, Idiomarina loihiensis, Alcanivorax borkumensis, Acinetobacter sp., Caulobacter crescentus, Alcaligenes eutrophus, Alcaligenes latus, Azotobacter vinlandii, Rhodococcus eutropha, Chromobacterium violaceum or Chromatium vinosum, preferably Pseudomonas putida strains, and more preferably Pseudomonas putida U.

9. The genetically engineered microorganism of any one of the preceding claims, wherein the microorganism is capable to produce PHA without the addition of an inducer molecule.

10. The genetically engineered microorganism of any one of the preceding claims, wherein the microorganism is capable to produce PHA in the form of a single intercellular granule.

11. The genetically engineered microorganism of any one of the preceding claims, wherein the microorganism is capable to produce a maximum content of PHA after 24 h upon exposure to modified MM medium containing sodium octanoate and preferably is also capable to maintain a PHA content, which is in a range of ±20% by weight of the maximum PHA content, for a time of at least 48 h.

12. A method for producing PHA comprising the following steps:
a. cultivating a microorganism of any one of claims 1 to 11 and
b. recovering PHAs from the culture medium.

13. The method according to claim 12, wherein said method does not involve or require the addition of an inducer molecule to initiate PHA overproduction and/or overproduction of PHA synthases in the microorganism and/or the addition of an antibiotic to prevent loss of the genetic modification.

14. The method according to claim 12 or 13, wherein the PHA is recovered by extraction with a ketone having 3 to 8 carbon atoms, preferably with acetone, at a temperature of 60° C. or less, preferably at 20 to 40° C.

15. Use of a microorganism of any one of claims 1 to 11 for the overproduction of medium- and/or long-chain-length PHA.


SEQUENCE LISTING (42 sequences)

SEQ ID NO: 1 - DNA, 20 bp, Artificial Sequence
  Forward Primer (5′ 3′) of 16s ribisomal DNA for Pseudomonas Putida KT2440 as a template
acgatccgtaactggtctga

SEQ ID NO: 2 - DNA, 20 bp, Artificial Sequence
  Reverse Primer (5′ 3′) of 16s ribisomal DNA for Pseudomonas Putida KT2440 as a template
ttcgcacctcagtgtcagta

SEQ ID NO: 3 - DNA, 22 bp, Artificial Sequence
  Forward Primer (5′ 3′) of Citrate synthase (glpA) PP_4194 for Pseudomonas Putida KT2440 as a template
gccgatttcatccagcatggtc

SEQ ID NO: 4 - DNA, 22 bp, Artificial Sequence
  Reverse Primer (5′ 3′) of Citrate synthase (glpA) PP_4194 for Pseudomonas Putida KT2440 as a template
tggaccggatcttcatcctcca

SEQ ID NO: 5 - DNA, 21 bp, Artificial Sequence
  Forward Primer (5′ 3′) of Ribosomal protein S12 (rpsL) PP_0449 for Pseudomonas Putida KT2440 as a template
ggcaactatcaaccagctggt

SEQ ID NO: 6 - DNA, 21 bp, Artificial Sequence
  Reverse Primer (5′ 3′) of Ribosomal protein S12 (rpsL) PP_0449 for Pseudomonas Putida KT2440 as a template
gctgtgctcttgcaggttgtg

SEQ ID NO: 7 - DNA, 21 bp, Artificial Sequence
  Forward Primer (5′ 3′) of Glyceraldehyde 3-phosphate dehydrogenase (gap-1) PP_1009 for Pseudomonas Putida KT2440 as a template
cttgaggttgacggtgaggtc

SEQ ID NO: 8 - DNA, 22 bp, Artificial Sequence
  Reverse Primer (5′ 3′) of Glyceraldehyde 3-phosphate dehydrogenase (gap-1) PP_1009 for Pseudomonas Putida KT2440 as a template
aggtgctgactgacgtttacca

SEQ ID NO: 9 - DNA, 23 bp, Artificial Sequence
  Forward Primer (5′ 3′) of Signal recognition particle protein Ffh (ffH) PP_1461 for Pseudomonas Putida KT2440 as a template
cggtagtcaaggatttcgtcaac

SEQ ID NO: 10 - DNA, 22 bp, Artificial Sequence
  Reverse Primer (5′ 3′) of Signal recognition particle protein Ffh (ffH) PP_1461 for Pseudomonas Putida KT2440 as a template
caccatcacgctctttttcttg

SEQ ID NO: 11 - DNA, 22 bp, Artificial Sequence
  Forward Primer (5′ 3′) of Rod shape-determining protein MreB (mreB) PP_0933 for Pseudomonas Putida KT2440 as a template
cgtgaagtgttcctgatcgaag

SEQ ID NO: 12 - DNA, 22 bp, Artificial Sequence
  Reverse Primer (5′ 3′) of Rod shape-determining protein MreB (mreB) PP_0933 for Pseudomonas Putida KT2440 as a template
ccgatttcctgcttgatacgtt

SEQ ID NO: 13 - DNA, 22 bp, Artificial Sequence
  Forward Primer (5′ 3′) of Cell division protein FtsZ (ftsZ) PP_1342 for Pseudomonas Putida KT2440 as a template
cggtatctccgacatcatcaag

SEQ ID NO: 14 - DNA, 22 bp, Artificial Sequence
  Reverse Primer (5′ 3′) of Cell division protein FtsZ (ftsZ) PP_1342 for Pseudomonas Putida KT2440 as a template
gagtactcacccagcgacaggt

SEQ ID NO: 15 - DNA, 22 bp, Artificial Sequence
  Forward Primer (5′ 3′) of Pyrroline-5- carboxylate reductase 1 (proC1) PP_3778 for Pseudomonas Putida KT2440 as a template
gcatttaccagccctttgaagc

SEQ ID NO: 16 - DNA, 22 bp, Artificial Sequence
  Reverse Primer (5′ 3′) of Pyrroline-5- carboxylate reductase 1 (proC1) PP_3778 for Pseudomonas Putida KT2440 as a template
caatgacgaaaggcaaatcgac

SEQ ID NO: 17 - DNA, 21 bp, Artificial Sequence
  Forward Primer (5′ 3′) of Pyrroline-5- carboxylate reductase 2 (proC2) PP_5095 for Pseudomonas Putida KT2440 as a template
ctcccaactgaccttgcagac

SEQ ID NO: 18 - DNA, 23 bp, Artificial Sequence
  Reverse Primer (5′ 3′) of Pyrroline-5- carboxylate reductase 2 (proC2) PP_5095 for Pseudomonas Putida KT2440 as a template
gctccttatttgcccagttgttc

SEQ ID NO: 19 - DNA, 19 bp, Artificial Sequence
  Forward Primer (5′ 3′) of PHA synthase 1 (phaC1) for Pseudomonas Putida U as a template
gcatgtggcccactttggc

SEQ ID NO: 20 - DNA, 19 bp, Artificial Sequence
  Reverse Primer (5′ 3′) of PHA synthase 1 (phaC1) for Pseudomonas Putida U as a template
cccaggttcttgcccactt

SEQ ID NO: 21 - DNA, 21 bp, Artificial Sequence
  Forward Primer (5′ 3′) of PHA depolymerase (phaZ) for Pseudomonas Putida U as a template
agcagtttgcccacgactacc

SEQ ID NO: 22 - DNA, 21 bp, Artificial Sequence
  Reverse Primer (5′ 3′) of PHA depolymerase (phaZ) for Pseudomonas Putida U as a template
ggtggatcttgtgcagccagt

SEQ ID NO: 23 - DNA, 21 bp, Artificial Sequence
  Forward Primer (5′ 3′) of PHA synthase 2 (phaC2) for Pseudomonas Putida U as a template
ggcaaccccaaggcctactac

SEQ ID NO: 24 - DNA, 21 bp, Artificial Sequence
  Reverse Primer (5′ 3′) of PHA synthase 2 (phaC2) for Pseudomonas Putida U as a template
ccgagcggtggataggtactg

SEQ ID NO: 25 - DNA, 23 bp, Artificial Sequence
  Forward Primer (5′ 3′) of Phasin PhaF (phaF) for Pseudomonas Putida U as a template
gtcagcttctcgatctgcttggt

SEQ ID NO: 26 - DNA, 24 bp, Artificial Sequence
  Reverse Primer (5′ 3′) of Phasin PhaF (phaF) for Pseudomonas Putida U as a template
gaagaagacggctgaagatgtagc

SEQ ID NO: 27 - DNA, 22 bp, Artificial Sequence
  Forward Primer (5′ 3′) of Phasin PhaI (phaI) for Pseudomonas Putida U as a template
ctctttgtcgatgcgtttcttg

SEQ ID NO: 28 - DNA, 22 bp, Artificial Sequence
  Reverse Printer (5′ 3′) of Phasin PhaI (phaI) for Pseudomonas Putida U as a template
catggccaaagtgattgtgaag

SEQ ID NO: 29 - DNA, 22 bp, Artificial Sequence
  Forward Printer (5′ 3′) of PhaD transcriptional regulator (phaD) for Pseudomonas Putida U as a template
gaacgtatccaccctggagatt

SEQ ID NO: 30 - DNA, 24 bp, Artificial Sequence
  Reverse Printer (5′ 3′) of PhaD transcriptional regulator (phaD) for Pseudomonas Putida U as a template
ataaggtgcaggaacagccagtag

SEQ ID NO: 31 - DNA, 25 bp, Artificial Sequence
  Forward Primer (5′ 3′) of Long-chain-fatty- acid-CoA ligase 1 (fadD1) for Pseudomonas Putida U as a template
cgtgatcaagtacgtgaagaagatg

SEQ ID NO: 32 - DNA, 23 bp, Artificial Sequence
  Reverse Primer (5′ 3′) of Long-chain-fatty- acid-CoA ligase 1 (fadD1) for Pseudomonas Putida U as a template
gtgaaggcgtagatgtggtacag

SEQ ID NO: 33 - DNA, 24 bp, Artificial Sequence
  Forward Primer (5′ 3′) of Long-chain-fatty- acid-CoA ligase 2 (fadD2) for Pseudomonas Putida U as a template
gctgtaccacatctatgccttcac

SEQ ID NO: 34 - DNA, 21 bp, Artificial Sequence
  Reverse Primer (5′ 3′) of Long-chain-fatty- acid-CoA ligase 2 (fadD2) for Pseudomonas Putida U as a te mplate
gccggagttggtgactttcag

SEQ ID NO: 35 - DNA, 16 bp, Artificial Sequence
  Forward Primer (5′ 3′) M13F
gtaaaacgacggccag

SEQ ID NO: 36 - DNA, 16 bp, Artificial Sequence
  Reverse Primer (5′ 3′) M13r
aggaaacagctatgac

SEQ ID NO: 37 - DNA, 24 bp, Artificial Sequence
  Forward Primer (5′ 3′) PhaC1
gaatcggttgtgaaactcatgctc

SEQ ID NO: 38 - DNA, 25 bp, Artificial Sequence
  Reverse Primer (5′ 3′) PhaC2-check-R
ccttgccatggaagtggtagtacag

SEQ ID NO: 39 - DNA, 21 bp, Artificial Sequence
  Forward Primer (5′ 3′) RT-phaZ F_PpU
agcagtttgcccacgactacc

SEQ ID NO: 40 - DNA, 21 bp, Artificial Sequence
  Reverse Primer (5′ 3′) RT-phaZ R_PpU
ggtggatcttgtgcagccagt

SEQ ID NO: 41 - DNA, 35 bp, Artificial Sequence
  Forward Primer (5′ 3′) phaZ-F-KpnI
ggggtacccccactttttcacgacagagtcgaacg

SEQ ID NO: 42 - DNA, 31 bp, Artificial Sequence
  Reverse Primer (5′ 3′) phaZ-R-XbaI
gctctagagcgcaacactccctcgtcttacc